(19) United States
(12) Patent Application Publication
(10) Pub. No.: US 2024/0392014 A1
(43) Pub. Date: Nov. 28, 2024

(54) AAV-MEDIATED EXPRESSION OF LONG-ACTING ANTI-CCR5 BINDING AGENTS FOR THE TREATMENT AND PREVENTION OF HIV

(71) Applicants: Oregon Health & Science University, Portland, OR (US); CytoDyn, Inc., Vancouver, WA (US)

(72) Inventors: Jonah Sacha, Portland, OR (US); Scott Kelly, Vancouver, WA (US); Helen Wu, Portland, OR (US); Gabriela Webb, Portland, OR (US)

(21) Appl. No.: 18/527,054

(22) Filed: Dec. 1, 2023

Related U.S. Application Data
(60) Provisional application No. 63/429,879, filed on Dec. 2, 2022.

(51) Int. Cl. C07K 16/28; A61K 39/00; A61K 48/00; A61P 31/18

(52) U.S. Cl. CPC C07K 16/2866; A61K 48/0033; A61P 31/18; A61K 2039/505

(57) ABSTRACT

The present disclosure provides recombinant adenovirus-associated viral vectors for the delivery of CCR5 binding agents with increased effector function and circulation half-life that are useful for treating and preventing HIV and methods of use thereof.

### PRIORITY CLAIM

The present application claims priority to U.S. Provisional Patent Application Ser. No. 63/429,879 filed Dec. 2, 2022 and titled “AAV-MEDIATED EXPRESSION OF LONG-ACTING ANTI-CCR5 BINDING AGENTS FOR THE TREATMENT AND PREVENTION OF HIV,” which is incorporated by reference herein in its entirety.

### GOVERNMENT SUPPORT

This invention was made with government support under grant no. 1R01AI154559-01 awarded by The National Institutes of Health. The government has certain rights in the invention.

### REFERENCE TO AN ELECTRONIC SEQUENCE LISTING

The contents of the electronic sequence listing (439_SeqListing.xml; Size: 31,428 bytes; and Date of Creation: Aug. 16, 2024) is herein incorporated by reference in its entirety.

### TECHNICAL FIELD

The present disclosure provides recombinant adenovirus-associated viral vectors for the delivery of CCR5 binding agents with increased effector function and circulation half-life that are useful for treating and preventing HIV and methods of use thereof.

### BACKGROUND

Currently, the most people ever in history are living with HIV due to continued new infections and greater access to antiretroviral treatment. To effectively slow the epidemic, approaches are needed to both suppress viral replication long-term and halt new infections.

Antiretroviral therapy (ART) is commonly used to treat HIV-positive individuals to suppress viral replication long-term. ART is also one of the most promising approaches to halt new HIV infections when used as pre-exposure prophylaxis (PrEP) to prevent HIV acquisition. HIV-positive individuals on prolonged ART experience non-HIV related morbidities such as liver dysfunction, cardiovascular disease, bone disorders, and lipodystrophy. Similarly, use of ART in HIV-negative individuals as PrEP results in unwanted side effects that reduce adherence. Therefore, the development of new user-friendly HIV suppression and PrEP regimens, particularly with simplified dosing regimens that lack unwanted negative side effects, is an urgent global health need.

CCR5 is the main HIV entry co-receptor, and thus represents a promising antiviral therapeutic target. Sexual transmission of HIV is due to the transmission of only one or two clones, and these founder viruses almost universally utilize CCR5, not CXCR4, as the co-receptor. The reliance of transmitted founder HIV clones on the CCR5 co-receptor explains the extremely high level of resistance to HIV infection exhibited by individuals homozygous for the CCR5Δ32 allele. Furthermore, the only two known cases of HIV cure occurred in the setting of allogeneic stem cell transplantation using a stem cell graft from a CCR5^{Δ32/Δ32} homozygous donor. These characteristics suggest CCR5 is an ideal target for (1) pre-exposure prophylaxis (PrEP) to prevent HIV acquisition, and (2) for long-term functional cure through control of HIV replication and plasma viremia.

To this end, the present disclosure provides recombinant adenovirus-associated viral vectors for the delivery of CCR5 binding agents with increased effector function and circulation half-life that are useful for treating and preventing HIV and methods of using the same.

### SUMMARY

The present disclosure provides recombinant adenovirus-associated viral (rAAV) vectors for the delivery of CCR5 binding agents with increased effector function and circulation half-life that are useful for treating and preventing HIV. Also provided are methods for the treatment and prevention of HIV by administering to the patient, an effective amount said rAAV vectors.

The present disclosure provides a recombinant adeno-associated virus (rAAV) vector comprising a heterologous nucleic acid encoding a CCR5 antibody comprising the following human IgG Fc amino acid substitutions: (i) M428L and N434S; (ii) L234A and L235A; and (iii) S131C wherein the antibody comprises: (a) a heavy chain variable region (VH) comprising a heavy chain complementary determining region 1 (HCDR1) of SEQ ID NO:11, a heavy chain complementary determining region 2 (HCDR2) of SEQ ID NO:12, and a heavy chain complementary determining region 3 (HCDR3) of SEQ ID NO:13; and (b) a light chain variable region (VL) comprising a light chain complementary determining region 1 (LCDR1) of SEQ ID NO:9, a light chain complementary determining region 2 (LCDR2) of SEQ ID NO:10, and a light chain complementary determining region 3 (LCDR3) of SEQ ID NO:11.

In some additional embodiments, the vector may include one or more of the following additional elements, in any combinations unless clearly mutually exclusive:
the human IgG Fc is a human IgG1 or IgG4;
the vector is a rAAV having a capsid selected from AAV1, AAV2, AAV3, AAV4, AAV5, AAV6, AAV7, AAV8, AAV9, AAV10, AAV11, or AAVrh10 and variants thereof; or
the vector is a rAAV having a capsid selected from AAV9 and variants thereof.

The present disclosure further provides a method of treating a HIV infection in a subject comprising administering to the subject an effective amount of a recombinant adeno-associated virus (rAAV) vector comprising a heterologous nucleic acid encoding a CCR5 antibody comprising the following human IgG Fc amino acid substitutions: (i) M428L and N434S; (ii) L234A and L235A; and (iii) S131C wherein the antibody comprises: (a) a heavy chain variable region (VH) comprising a heavy chain complementary determining region 1 (HCDR1) of SEQ ID NO:11, a heavy chain complementary determining region 2 (HCDR2) of SEQ ID NO: 12, and a heavy chain complementary determining region 3 (HCDR3) of SEQ ID NO:13; and (b) a light chain variable region (VL) comprising a light chain complementary determining region 1 (LCDR1) of SEQ ID NO: 9, a light chain complementary determining region 2 (LCDR2) of SEQ ID NO:10, and a light chain complementary determining region 3 (LCDR3) of SEQ ID NO:11.

In some additional embodiments, the method may include one or more of the following additional elements, in any combinations unless clearly mutually exclusive:
the vector is administered at a dose of at least 1×10¹² genomes/kg;
the vector is administered at a dose of at least 2×10¹² genomes/kg;
the percentage of CCR5 receptors occupied by the CCR5 antibody on CCR5+ CD4+ T cells is increased;
the period of time in which CCR5 receptors are occupied by the CCR5 antibody on CCR5+ CD4+ T cells is extended;
the period of time in which CCR5 receptors are fully occupied by the CCR5 antibody on CCR5+ CD4+ T cells in the blood is at least 13 weeks;
the period of time in which CCR5 receptors are fully occupied by the CCR5 antibody on CCR5+ CD4+ T cells in the blood is at least 38 weeks;
the period of time in which CCR5 receptors are fully occupied by the CCR5 antibody on CCR5+ CD4+ T cells in the mesenteric lymph nodes is at least 13 weeks;
the period of time in which CCR5 receptors are fully occupied by the CCR5 antibody on CCR5+ CD4+ T cells in the mesenteric lymph nodes is at least 38 weeks;
the period of time in which CCR5 receptors are fully occupied by the CCR5 antibody on CCR5+ CD4+ T cells in the spleen is at least 13 weeks; or
the period of time in which CCR5 receptors are fully occupied by the CCR5 antibody on CCR5+ CD4+ T cells in the spleen is at least 38 weeks.

### BRIEF DESCRIPTION OF THE DRAWINGS

FIG. 1 shows a bicistronic single-stranded AAV expression cassette used to express the MacLS sequence. The cassette consists of a CMV promoter, SV40 intron, signal peptides (SP), variable heavy (VH) and variable light (VL) chains of leronlimab, rhesus IgG4 constant heavy (HC) and light (CL) chain coding sequences, Furin peptide with SGSG linker peptide, F2A peptide, miR-142 binding site (bs) sequence, and the SV40 polyA signal, flanked by AAV ITRs. The HC contains the M428L/N434S (“LS”) half-life extending mutations.

FIGS. 2A-2F show the ability of the leronlimab-delivering AAV to transduce myocytes and produce leronlimab in an in vivo rhesus macaque model of chronic simian HIV (SHIV) infection. An AAV9 encoding human Fc leronlimab (AAV9-huLeron) was administered at 2×10¹² AAV genomes/kg of to a Mauritian cynomolgus macaque (MCM) chronically infected with the CCR5-tropic virus SHIV_{SF162P3}. Dexamethasone (at −12, −1, and 5 hours post-AAV) and tacrolimus (daily, days-8 to 28 post-AAV) were also administered. Leronlimab in plasma (FIG. 2A, reported in μg/ml) and anti-drug antibody in plasma (FIG. 2B, reported as reciprocal dilution) are shown. CCR5 receptor occupancy in blood CCR5+ CD4+ T cells (left axis) versus SHIV viral load in plasma (right axis) is shown in FIG. 2C. FIG. 2D shows CCR5 receptor occupancy of CCR5+CD4+ T cells in blood, mesenteric lymph nodes (“MesLN”), and spleen at weeks 13 and 38 post-AAV9-huLeron. FIG. 2E shows cell-associated SHIV DNA in the blood, MesLN, and spleen at weeks 2, 13, 38 and 53 post-AAV9-huLeron. FIG. 2F shows cell-associated SHIV DNA in the blood, MesLN, and spleen at weeks 2, 13, 38 and 53 post-AAV9-huLeron.

FIGS. 3A-3C show the ability of the macaque IgG4 LS-delivering AAV (Fc LALA LS SC leronlimab (AAV9-macLSLeron)) to control HIV replication and mediate a functional cure in an in vivo rhesus macaque model of chronic simian HIV (SHIV) infection. One SHIV_{AD8EOM}-infected rhesus macaque received 2×10¹² genomes/kg of AAV9-macLS. Leronlimab in plasma (FIG. 3A, reported in μg/ml), anti-drug antibody in plasma (FIG. 3B, reported as reciprocal dilution), and CCR5 receptor occupancy in blood (left axis) versus viral load in plasma (right axis) are shown (FIG. 3C).

FIGS. 4A-4F show further tests of the ability of the IgG4 LS-delivering AAAV9-macLSLeron to control HIV replication and mediate a functional cure in an in vivo rhesus macaque model of chronic simian HIV (SHIV) infection using three SHIV_{AD8EOM}-infected rhesus macaques, two of which (designated 38073 and 37660) received 2×10¹² genomes/kg of AAV9-macLS, and one of which received only 1×10¹² genomes/kg of AAV9-macLS. Leronlimab in plasma (FIG. 4A, reported in μg/ml), anti-drug antibody in plasma (FIG. 4B, reported as reciprocal dilution), CD4+ T cell CCR5 receptor occupancy in by leronlimab in blood, peripheral lymph nodes (“PerhphLN”), MesLn, and spleen at weeks 23-26 post AAV9-huLeron (FIG. 4C, macaque 38073=left bar for each sample type, macaque 37660=middle bar for each sample type, and macaque 37939=right bar for each sample type), and CCR5 receptor occupancy in blood (left axis and gray-scale line in upper portion of figure) versus viral load in plasma (right axis and black line in lower portion of figure) for macaque 38073 (FIG. 4D), macaque 37660 (FIG. 4E) and macaque 37939 (FIG. 4F) are shown. SHIV LOQ was 15 copies/mL.

FIGS. 5A-5F show test results for of the ability of the IgG4 LS-delivering AAAV9-macLSLeron to control HIV replication and mediate a functional cure in an in vivo rhesus macaque model of chronic simian HIV (SHIV) infection in one SHIV_{AD8EOM}-infected rhesus macaque which received 2×10¹² genomes/kg of AAV9-macLS. Leronlimab in plasma (FIG. 5A, reported in μg/ml), anti-drug antibody in plasma (FIG. 5B, reported as reciprocal dilution), CD4+ T cell CCR5 receptor occupancy in by leronlimab in blood (left axis and gray-scale line in upper portion of figure) versus SHIV plasma viral load (right axis and black line in lower portion of figure) (FIG. 5C), CD4+ T cell CCR5 receptor occupancy by leronlimab in blood, MesLN and spleen at pre-AAV (left bar for each sample type), week 10 post-AAV (second-from-left bar for each sample type), week 63 post-AAV (second-from-right bar for each sample type) and week 87 post-AAV (right bar for each sample type) (FIG. 4D), cell-associated SHIV DNA in MesLN or spleen at pre-AAV (left bar for each sample type), week 10 post-AAV (second-from-left bar for each sample type), week 63 post-AAV (second-from-right bar for each sample type) and week 87 post-AAV (right bar for each sample type) (FIG. 4E), and cell-associated SHIV RNA in MesLN or spleen at pre-AAV (left bar for each sample type), week 10 post-AAV (second-from-left bar for each sample type), week 63 post-AAV (second-from-right bar for each sample type) and week 87 post-AAV (right bar for each sample type) (FIG. 4F) are shown.

### DETAILED DESCRIPTION

The present disclosure provides recombinant adenovirus-associated viral vectors for the delivery of CCR5 binding agents with increased effector function and circulation half-life that are useful for treating and preventing HIV and methods of use thereof.

Prior to setting forth this disclosure in more detail, it may be helpful to an understanding thereof to provide definitions of certain terms to be used herein. Unless defined otherwise, all technical and scientific terms used herein have the same meaning as is commonly understood by one of skill in the art to which this invention belongs. Additional definitions are set forth throughout this disclosure.

In the present description, the term “about” means ±20% of the indicated range, value, or structure, unless otherwise indicated. The term “consisting essentially of” limits the scope of a claim to the specified materials or steps and those that do not materially affect the basic and novel characteristics of the claimed invention. It should be understood that the terms “a” and “an” as used herein refer to “one or more” of the enumerated components. The use of the alternative (e.g., “or”) should be understood to mean either one, both, or any combination thereof of the alternatives. As used herein, the terms “include” and “have” are used synonymously, which terms and variants thereof are intended to be construed as non-limiting. The term “comprise” means the presence of the stated features, integers, steps, or components as referred to in the claims, but that it does not preclude the presence or addition of one or more other features, integers, steps, components, or groups thereof. Any ranges provided herein include all the values and narrower ranges in the ranges.

As used herein, “chemokine” refers to a low-molecular weight cytokine that can stimulate recruitment of leukocytes. Chemokines have cysteine residues in conserved locations that are key to forming their 3-dimensional shape. Chemokines may be classified into four main subfamilies: Cys-Cys (C-C), Cys-X-Cys (CXC), CX₃C, and XC depending on the spacing of their first two amino terminal cysteine residues. Chemokines may also be grouped according to their function, such as whether they are inflammatory or homeostatic. There are 47 known chemokines, including but not limited to CCL5 (also known as RANTES), MIP-1α, MIP-1β, or SDF-1, or another chemokine which has similar activity.

As used herein, “C-C chemokine receptor 5,” also known as “CCR5” or “CD195” refers to a G protein-coupled receptor expressed on lymphocytes (e.g., NK cells, B cells, T cells), monocytes, dendritic cells, eosinophils, and microglia, which functions as a chemokine receptor for the C-C chemokine group. CCR5's cognate ligands include CCL3, CCL4, CCL3L1, and CCL5. In some embodiments, CCR5 refers to human CCR5. In some embodiments, CCR5 refers to a protein having an amino acid sequence provided in NCBI Reference Sequence: NP_000570.1 (SEQ ID NO: 15).

As used herein, “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, γ-carboxyglutamate, and O-phosphoserine. Amino acid analogs refer to compounds that have the same basic chemical structure as a naturally occurring amino acid, i.e., an α-carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refer to chemical compounds that have a structure that is different from the general chemical structure of an amino acid, but that functions in a manner similar to a naturally occurring amino acid.

As used herein, “mutation” refers to a change in the sequence of a nucleic acid molecule or polypeptide molecule as compared to a reference or wild-type nucleic acid molecule or polypeptide molecule, respectively. A mutation can result in several different types of change in sequence, including substitution, insertion or deletion of nucleotide(s) or amino acid(s).

As used herein, “protein” or “polypeptide” as used herein refers to a compound made up of amino acid residues that are covalently linked by peptide bonds. The term “protein” may be synonymous with the term “polypeptide” or may refer, in addition, to a complex of two or more polypeptides. A polypeptide may further contain other components (e.g., covalently bound), such as a tag, a label, a bioactive molecule, or any combination thereof. In certain embodiments, a polypeptide may be a fragment. As used herein, a “fragment” means a polypeptide that is lacking one or more amino acids that are found in a reference sequence. A fragment can comprise a binding domain, antigen, or epitope found in a reference sequence. A fragment of a reference polypeptide can have at least about 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99%, or more of amino acids of the amino acid sequence of the reference sequence.

As described herein, a “variant” polypeptide species has one or more non-natural amino acids, one or more amino acid substitutions, one or more amino acid insertions, one or more amino acid deletions, or any combination thereof at one or more sites relative to a reference polypeptide as presented herein. In certain embodiments, “variant” means a polypeptide having a substantially similar activity (e.g., enzymatic function, immunogenicity) or structure relative to a reference polypeptide). A variant of a reference polypeptide can have at least about 40%, 45%, 50%, 55%, 60%, 65%, 70%, 75%, 80%, 85%, 90%, 91%, 92%, 93%, 94%, 95%, 96%, 97%, 98%, 99%, or more sequence identity to the amino acid sequence for the reference polypeptide as determined by sequence alignment programs and parameters known in the art. The variant can result from, for example, a genetic polymorphism or human manipulation. Conservative substitutions of amino acids are well known and may occur naturally or may be introduced when a protein is recombinantly produced. Amino acid substitutions, deletions, and additions may be introduced into a protein using mutagenesis methods known in the art (see, e.g., Sambrook et al., Molecular Cloning: A Laboratory Manual, 3d ed., Cold Spring Harbor Laboratory Press, NY, 2001). Oligonucleotide-directed site-specific (or segment specific) mutagenesis procedures may be employed to provide an altered polynucleotide that has particular codons altered according to the substitution, deletion, or insertion desired. Alternatively, random or saturation mutagenesis techniques, such as alanine scanning mutagenesis, error prone polymerase chain reaction mutagenesis, and oligonucleotide-directed mutagenesis may be used to prepare polypeptide variants (see, e.g., Sambrook et al., supra).

A “conservative substitution” refers to amino acid substitutions that do not significantly affect or alter binding characteristics of a particular protein. Generally, conservative substitutions are ones in which a substituted amino acid residue is replaced with an amino acid residue having a similar side chain. Conservative substitutions include a substitution found in one of the following groups: Group 1: Alanine (Ala or A), Glycine (Gly or G), Serine (Ser or S), Threonine (Thr or T); Group 2: Aspartic acid (Asp or D), Glutamic acid (Glu or Z); Group 3: Asparagine (Asn or N), Glutamine (Gln or Q); Group 4: Arginine (Arg or R), Lysine (Lys or K), Histidine (His or H); Group 5: Isoleucine (Ile or I), Leucine (Leu or L), Methionine (Met or M), Valine (Val or V); and Group 6: Phenylalanine (Phe or F), Tyrosine (Tyr or Y), Tryptophan (Trp or W). Additionally or alternatively, amino acids can be grouped into conservative substitution groups by similar function, chemical structure, or composition (e.g., acidic, basic, aliphatic, aromatic, or sulfur-containing). For example, an aliphatic grouping may include, for purposes of substitution, Gly, Ala, Val, Leu, and Ile. Other conservative substitutions groups include: sulfur-containing: Met and Cysteine (Cys or C); acidic: Asp, Glu, Asn, and Gln; small aliphatic, nonpolar or slightly polar residues: Ala, Ser, Thr, Pro, and Gly; polar, negatively charged residues and their amides: Asp, Asn, Glu, and Gln; polar, positively charged residues: His, Arg, and Lys; large aliphatic, nonpolar residues: Met, Leu, Ile, Val, and Cys; and large aromatic residues: Phe, Tyr, and Trp. Additional information can be found in Creighton (1984) Proteins, W.H. Freeman and Company.

The terms “identical” or “percent identity,” in the context of two or more polypeptide or nucleic acid molecule sequences, means two or more sequences or subsequences that are the same or have a specified percentage of amino acid residues or nucleotides that are the same over a specified region (e.g., 60%, 65%, 70%, 75%, 80%, 85%, 90%, 91%, 92%, 93%, 94%, 95%, 96%, 97%, 98%, 99%, or 100% identity), when compared and aligned for maximum correspondence over a comparison window, or designated region, as measured using methods known in the art, such as a sequence comparison algorithm, by manual alignment, or by visual inspection. The algorithm used herein for determining percent sequence identity and sequence similarity is the BLAST 2.0 algorithm, as described in Altschul et al. “Gapped BLAST and PSI-BLAST: a new generation of protein database search programs,” Nucleic Acids Res. 2007, 25, 3389-3402. Within the context of this disclosure, it will be understood that where sequence analysis software is used for analysis, the results of the analysis are based on the “default values” of the program referenced. “Default values” mean any set of values or parameters which originally load with the software when first initialized.

As used herein, a “fusion protein” comprises a single chain polypeptide having at least two distinct domains, wherein the domains are not naturally found together in a protein. A nucleic acid molecule encoding a fusion protein may be constructed using PCR, recombinantly engineered, or the like, or such fusion proteins can be made synthetically. A fusion protein may further contain other components (e.g., covalently bound), such as a tag, linker, transduction marker, or bioactive molecule.

A “nucleic acid molecule” or “polynucleotide” refers to a polymeric compound containing nucleotides that are covalently linked by 3′-5′ phosphodiester bonds. Nucleic acid molecules include polyribonucleic acid (RNA), polydeoxyribonucleic acid (DNA), which includes genomic DNA, mitochondrial DNA, cDNA, or vector DNA. A nucleic acid molecule may be double stranded or single stranded, and if single stranded, may be the coding strand or non-coding (anti-sense strand). A nucleic acid molecule may contain natural subunits or non-natural subunits. A nucleic acid molecule encoding an amino acid sequence includes all nucleotide sequences that encode the same amino acid sequence. Some versions of the nucleotide sequences may also include intron(s) to the extent that the intron(s) would be removed through co- or post-transcriptional mechanisms. In other words, different nucleotide sequences may encode the same amino acid sequence as the result of the redundancy or degeneracy of the genetic code, or by splicing.

Variants of the polynucleotides of this disclosure are also contemplated. Variant polynucleotides are at least 80%, 85%, 90%, 95%, 99%, or 99.9% identical to a reference polynucleotide as described herein, or that hybridizes to a reference polynucleotide of defined sequence under stringent hybridization conditions of 0.015M sodium chloride, 0.0015M sodium citrate at about 65°−68° C. or 0.015M sodium chloride, 0.0015M sodium citrate, and 50% formamide at about 42° C. The polynucleotide variants retain the capacity to encode an immunoglobulin-like binding protein or antigen-binding fragment thereof having the functionality described herein.

The term “isolated” means that the material is removed from its original environment (e.g., the natural environment if it is naturally occurring). For example, a naturally occurring polynucleotide or polypeptide present in a living animal is not isolated, but the same polynucleotide or polypeptide, separated from some or all of the co-existing materials in the natural system, is isolated. Such polynucleotide could be part of a vector and/or such polynucleotide or polypeptide could be part of a composition (e.g., a cell lysate), and still be isolated in that such vector or composition is not part of the natural environment for the nucleic acid or polypeptide.

As used herein, the term “engineered,” “recombinant,” or “non-natural” refers to an organism, microorganism, cell, nucleic acid molecule, or vector that includes at least one genetic alteration or has been modified by introduction of an exogenous or heterologous nucleic acid molecule, wherein such alterations or modifications are introduced by genetic engineering (i.e., human intervention). Genetic alterations include, for example, modifications introducing expressible nucleic acid molecules encoding functional RNA, proteins, fusion proteins or enzymes, or other nucleic acid molecule additions, deletions, substitutions, or other functional disruption of a cell's genetic material. Additional modifications include, for example, non-coding regulatory regions in which the modifications alter expression of a polynucleotide, gene, or operon.

As used herein, “heterologous” or “exogenous” nucleic acid molecule, construct or sequence refers to a nucleic acid molecule or portion of a nucleic acid molecule that is not native to a host cell, but may be homologous to a nucleic acid molecule or portion of a nucleic acid molecule from the host cell. The source of the heterologous or exogenous nucleic acid molecule, construct or sequence may be from a different genus or species. In certain embodiments, a heterologous or exogenous nucleic acid molecule is added (i.e., not endogenous or native) to a host cell or host genome by, for example, conjugation, transformation, transfection, electroporation, or the like, wherein the added molecule may integrate into the host genome or exist as extra-chromosomal genetic material (e.g., as a plasmid or other form of self-replicating vector), and may be present in multiple copies. In addition, “heterologous” refers to a non-native enzyme, protein, or other activity encoded by an exogenous nucleic acid molecule introduced into the host cell, even if the host cell encodes a homologous protein or activity.

As used herein, the term “endogenous” or “native” refers to a gene, protein, or activity that is normally present in a host cell. Moreover, a gene, protein or activity that is mutated, overexpressed, shuffled, duplicated or otherwise altered as compared to a parent gene, protein or activity is still considered to be endogenous or native to that particular host cell. For example, an endogenous control sequence from a first gene (e.g., promoter, translational attenuation sequences) may be used to alter or regulate expression of a second native gene or nucleic acid molecule, wherein the expression or regulation of the second native gene or nucleic acid molecule differs from normal expression or regulation in a parent cell.

As used herein, the term “expression”, refers to the process by which a polypeptide is produced based on the encoding sequence of a nucleic acid molecule, such as a gene. The process may include transcription, post-transcriptional control, post-transcriptional modification, translation, post-translational control, posttranslational modification, or any combination thereof. An expressed nucleic acid molecule is typically operably linked to an expression control sequence (e.g., a promoter).

As used herein, the term “operably linked” refers to the association of two or more nucleic acid molecules on a single nucleic acid fragment so that the function of one is affected by the other. For example, a promoter is operably linked with a coding sequence when it is capable of affecting the expression of that coding sequence (i.e., the coding sequence is under the transcriptional control of the promoter). “Unlinked” means that the associated genetic elements are not closely associated with one another and the function of one does not affect the other.

As used herein, “expression vector” refers to a DNA construct containing a nucleic acid molecule that is operably linked to a suitable control sequence capable of effecting the expression of the nucleic acid molecule in a suitable host. Such control sequences include a promoter to effect transcription, an optional operator sequence to control such transcription, a sequence encoding suitable mRNA ribosome binding sites, and sequences which control termination of transcription and translation. The vector may be a plasmid, a phage particle, a virus, or simply a potential genomic insert. Once transformed into a suitable host, the vector may replicate and function independently of the host genome, or may, in some instances, integrate into the genome itself. In the present specification, “plasmid,” “expression plasmid,” “virus” and “vector” are often used interchangeably.

As used herein, the term “microRNA” refers to a major class of biomolecules involved in control of gene expression. For example, in human heart, liver or brain, miRNAs play a role in tissue specification or cell lineage decisions in addition, miRNAs influence a variety of processes, including early development, cell proliferation and ceil death, and apoptosis and fat metabolism. The large number of miRNA genes, the diverse expression patterns, and the abundance of potential miRNA targets suggest that miRNAs may be a significant source of genetic diversity.

A mature miRNA is typically an 8-25 nucleotide non-coding RNA that regulates expression of an mRNA including sequences complementary to the miRNA. These small RNA molecules are known to control gene expression by regulating the stability and/or translation of mRNAs. For example, miRNAs bind to the 3′ UTR of target mRNAs and suppress translation. MiRNAs may also bind to target mRNAs and mediate gene silencing through the RNAi pathway. MiRNAs may also regulate gene expression by causing chromatin condensation.

A miRNA silences translation of one or more specific mRNA molecules by binding to a miRNA recognition element (MRE,) which is defined as any sequence that directly base pairs with and interacts with the miRNA somewhere on the mRNA transcript. Often, the MRE is present in the 3′ untranslated region (UTR) of the mRNA, but it may also be present in the coding sequence or in the 5′ UTR. MREs are not necessarily perfect complements to miRNAs, usually having only a few bases of complementarity to the miRNA and often containing one or more mismatches within those bases of complementarity. The MRE may be any sequence capable of being bound by a miRNA sufficiently that the translation of a gene to which the MRE is operably linked (such as a CMV gene that is essential or augmenting for growth in vivo) is repressed by a miRNA silencing mechanism such as the RISC.

As used herein, the term “host” refers to a cell (e.g., T cell, Chinese Hamster Ovary (CHO) cell, HEK293 cell, B cell, or the like) or microorganism targeted for genetic modification with a heterologous nucleic acid molecule to produce a polypeptide of interest (e.g., a CCR5 antibody of the present disclosure). In certain embodiments, a host cell may optionally already possess or be modified to include other genetic modifications that confer desired properties related or unrelated to, e.g., biosynthesis of the heterologous protein (e.g., inclusion of a detectable marker; deleted, altered or truncated endogenous BCR).

As described herein, more than one heterologous nucleic acid molecule can be introduced into a host cell as separate nucleic acid molecules, as a plurality of individually controlled genes, as a polycistronic nucleic acid molecule (e.g., a heavy chain and a light chain of an antibody), as a single nucleic acid molecule encoding a protein (e.g., a heavy chain of an antibody), or any combination thereof. When two or more heterologous nucleic acid molecules are introduced into a host cell, it is understood that the two or more heterologous nucleic acid molecules can be introduced as a single nucleic acid molecule (e.g., on a single vector), on separate vectors, integrated into the host chromosome at a single site or multiple sites, or any combination thereof. The number of referenced heterologous nucleic acid molecules or protein activities refers to the number of encoding nucleic acid molecules or the number of protein activities, not the number of separate nucleic acid molecules introduced into a host cell.

As used herein, the term “introduced” in the context of inserting a nucleic acid sequence into a cell, means “transfection”, or “transformation” or “transduction” and includes reference to the incorporation of a nucleic acid sequence into a eukaryotic or prokaryotic cell wherein the nucleic acid molecule may be incorporated into the genome of a cell (e.g., chromosome, plasmid, plastid, or mitochondrial DNA), converted into an autonomous replicon, or transiently expressed (e.g., transfected mRNA).

“Treat” or “treatment” or “ameliorate” refers to medical management of a disease, disorder, or condition of a patient (e.g., a human or non-human mammal, such as a primate, horse, cat, dog, goat, mouse, or rat). In general, an appropriate dose or treatment regimen comprising leronlimab is administered in an amount sufficient to elicit a therapeutic effect or therapeutic benefit. Treating an HIV infection in a subject refers to slowing, stopping or reversing the progression of an HIV disorder in the subject. In a preferred embodiment, “treating” refers to reversing the progression to the point of eliminating the disorder. As used herein, “treating” also means reducing the number of viral infections, reducing the number of infectious viral particles, reducing the number of virally infected cells, or ameliorating symptoms associated with HIV. Reducing viral load in a subject is one embodiment of treating the subject.

A prophylactic treatment meant to “prevent” a disease or condition (e.g., HIV infection in a patient) is a treatment administered to a patient who does not exhibit signs of a disease or exhibits only early signs, for the purpose of decreasing the risk of developing pathology or further advancement of the early disease. Such a prophylactic treatment may be referred to as “pre-exposure prophylaxis” (PrEP). For example, if an individual at risk of contracting HIV is treated with the methods of the present disclosure and does not contract HIV, then the disease has been prevented, at least over a period of time, in that individual.

A “therapeutically effective amount” or “effective amount” of leronlimab refers to an amount of leronlimab sufficient to result in a therapeutic effect, including improved clinical outcome; lessening or alleviation of symptoms associated with a disease; modulating immune response to lessen, reduce, or dampen counterproductive inflammatory cytokine activity; modulating immune response to normalize counterproductive inflammatory cytokine activity; decreased occurrence of symptoms; improved quality of life; longer disease-free status; diminishment of extent of disease, stabilization of disease state; delay of disease progression; remission; survival; or prolonged survival in a statistically significant manner. When referring to an individual active ingredient or a cell expressing a single active ingredient, administered alone, a therapeutically effective amount refers to the effects of that ingredient or cell expressing that ingredient alone. When referring to a combination, a therapeutically effective amount refers to the combined amounts of active ingredients or combined adjunctive active ingredient with a cell expressing an active ingredient that results in a therapeutic effect, whether administered serially or simultaneously.

The term “pharmaceutically acceptable excipient or carrier” or “physiologically acceptable excipient or carrier” refer to biologically compatible vehicles, e.g., physiological saline, which are described in greater detail herein, that are suitable for administration to a human or other non-human mammalian patient and generally recognized as safe or not causing a serious adverse event.

The terms “CCR5 receptor occupancy” and “CCR5 RO” refer to the percentage of CCR5 RO on the surface of CD4+ T cells. An equation is used to measure unoccupied CCR5 receptors by using Pacific Blue-conjugated Leronlimab (termed Leronlimab-PB). CCR5 RO is defined as the percentage of cells CCR5+ (measured by non-competing alternative clone 3A9 or J418) and Leronlimab+ (measured by anti-human IgG4 or anti-rhesus IgG4, depending in the leronlimab version used) divided by the percentage of cells CCR5+ and Leronlimab+ (measured by the sum of anti-human or rhesus IgG4 and Leronlimab-PB) cells following incubation with a saturating concentration of Leronlimab-PB.

Additional definitions are provided in the sections below.

### CCR5 Binding Agents

The present disclosure provides for use of a CCR5 binding agent, e.g., leronlimab, or antigen binding fragment thereof, in the prevention and treatment of HIV. CCR5 binding agents for use in the present disclosure are inhibitors of CCR5 activity induced by CCL5 binding. The term “inhibit” or “inhibitor” refers to a diminishing, blunting, reduction, masking, interrupting, blocking, mitigation, or slowing directly or indirectly, in the expression, amount or activity of a target or signaling pathway relative to (1) a control, endogenous or reference target or pathway, or (2) the absence of a target or pathway, wherein the diminishing, blunting, reduction, masking, interrupting, blocking, mitigation, or slowing is statistically, biologically, or clinically significant. For example, an inhibitor of CCR5 may diminish, blunt, reduce, mask, interrupt, block, mitigate, or slow CCR5 signaling activity induced by CCL5 binding by about 25%, 30%, 35%, 40%, 45%, 50%, 55%, 60%, 65%, 70%, 75%, 80%, 85%, 86%, 87%, 88%, 89%, 90%, 91%, 92%, 93%, 94%, 95%, 96%, 97%, 98%, 99%, or more as compared to untreated CCR5. CCR5 activity induced by CCL5 binding may be measured by detecting, for example, a decrease in cAMP, cell migration, or both.

The CCR5 receptor is a C-C chemokine G-coupled protein receptor expressed on lymphocytes (e.g., NK cells, B cells), monocytes, monocytes, dendritic cells, a subset of T cells, etc. The extracellular portions represent potential targets for antibodies targeting CCR5, and comprise an amino-terminal domain (Nt) and three extracellular loops (ECL1, ECL2, and ECL3). The extracellular portions of CCR5 comprise just 90 amino acids distributed over four domains. The largest of these domains are at the Nt and ECL2 at approximately 30 amino acids each (Olson et al., Curr. Opin. HIV AIDS, March, 4(2): 104-111 (2009)).

The CCR5 receptor binds to a chemokine known as CCL5 (C-C chemokine ligand 5), which is an inflammatory chemokine that plays an important role in immunologic mechanisms such as controlling cell recruitment and activation in basal and inflammatory circumstances. CCL5 acts as a key regulator of CCR5+ cell (e.g., monocyte and T cell) migration to inflammatory sites, directing migration of monocytes and T cells to damaged or infected sites. CCR5 also plays a crucial role in differentiation and activation of CD8+ T cells. Many biologic effects of chemokines are mediated by their interaction with chemokine receptors on cell surfaces. The most relevant known receptor for CCL5 is the CCR5 receptor; however, CCR1 and CCR3 are also known CCL5 receptors and CCR4 and CD44 are auxiliary receptors. Tamamis et al., Elucidating a Key Anti-HIV-1 and Cancer-Associated Axis: The Structure of CCL5 (Rantes) in Complex with CCR5, SCIENTIFIC REPORTS, 4: 5447 (2014).

The formation of the CCL5 ligand and CCR5 receptor complex causes a conformational change in the receptor that activates the subunits of the G-protein, inducing signaling and leading to changed levels of cyclic AMP (CAMP), inositol triphosphate, intracellular calcium and tyrosine kinase activation. These signaling events cause cell polarization and translocation of the transcription factor NF-kB, which results in the increase of phagocytic ability, cell survival, and transcription of proinflammatory genes.

CCR5 binding agents include, but are not limited to, small molecules, antibodies or antigen binding fragments thereof, proteins, peptides, nucleic acids, and aptamers.

In some embodiments, a CCR5 binding agent is an anti-CCR5 antibody or antigen-binding fragment thereof that specifically binds to CCR5, e.g., to an epitope on CCR5. The term “epitope” or “antigenic epitope” includes any molecule, structure, amino acid sequence, or protein determinant that is recognized and specifically bound by a cognate binding molecule, such as an immunoglobulin, T cell receptor (TCR), chimeric antigen receptor, or other binding molecule, domain or protein. Epitopic determinants generally contain chemically active surface groupings of molecules, such as amino acids or sugar side chains, and can have specific three dimensional structural characteristics, as well as specific charge characteristics.

As used herein, “specifically binds” or “specific for” may in some embodiments refer to an association or union of a binding protein (e.g., an anti-CCR5 antibody) or a binding domain (or fusion protein thereof) to a target molecule with an affinity or Kₐ (i.e., an equilibrium association constant of a particular binding interaction with units of 1/M) equal to or greater than 10⁵ M⁻¹ (which equals the ratio of the on-rate [kₒₙ] to the off-rate [k_{off}] for this association reaction), while not significantly associating or uniting with any other molecules or components in a sample. Binding domains (or fusion proteins thereof) may be classified as “high affinity” binding domains (or fusion proteins thereof) and “low affinity” binding domains (or fusion proteins thereof). “High affinity” binding domains refer to those binding domains with a Kₐ of at least 10⁸ M⁻¹, at least 10⁹ M⁻¹, at least 10¹⁰ M⁻¹, at least 10¹¹ M⁻¹, at least 10¹² M⁻¹, or at least 10¹³ M⁻¹, preferably at least 10⁸ M⁻¹ or at least 10⁹ M⁻¹. “Low affinity” binding domains refer to those binding domains with a Kₐ of up to 10⁸ M⁻¹, up to 10⁷ M⁻¹, up to 10⁶ M⁻¹, up to 10⁵ M⁻¹. Alternatively, affinity may be defined as an equilibrium dissociation constant (K_{D}) of a particular binding interaction with units of M (e.g., 10⁻⁵ M to 10⁻¹³ M), (which equals the ratio of the off-rate [k_{off}] to the on-rate [kₒₙ] for this association reaction).

A variety of assays are known for identifying binding domains of the present disclosure that specifically bind a particular target, as well as determining binding domain or fusion protein affinities, such as Western blot, ELISA, analytical ultracentrifugation, spectroscopy and surface plasmon resonance (Biacore®) analysis (see, e.g., Scatchard et al., Ann. N.Y. Acad. Sci. 51:660, 1949; Wilson, Science 295:2103, 2002; Wolff et al., Cancer Res. 53:2560, 1993; and U.S. Pat. Nos. 5,283,173, 5,468,614, or the equivalent).

Terms understood by those in the art of antibody technology are each given the meaning acquired in the art, unless expressly defined differently herein. The term “antibody” refers to an intact antibody comprising at least two heavy (H) chains and two light (L) chains inter-connected by disulfide bonds, as well as any antigen-binding portion or fragment of an intact antibody, such as an scFv, Fab, or Fab′2 fragment, that has or retains the ability to bind to the antigen target molecule recognized by the intact antibody. Thus, the term “antibody” herein is used in the broadest sense and includes polyclonal and monoclonal antibodies, including intact antibodies and functional (antigen-binding) antibody fragments thereof, including fragment antigen binding (Fab) fragments, F(ab′)2 fragments, Fab′ fragments, Fv fragments, recombinant IgG (rIgG) fragments, single chain antibody fragments, including single chain variable fragments (scFv), and single domain antibodies (e.g., sdAb, sdFv, nanobody). The term encompasses genetically engineered and/or otherwise modified forms of immunoglobulins, such as intrabodies, peptibodies, chimeric antibodies, fully human antibodies, humanized antibodies, and heteroconjugate antibodies, multispecific, e.g., bispecific antibodies, diabodies, triabodies, tetrabodies, tandem di-scFv, and tandem tri-scFv. Unless otherwise stated, the term “antibody” should be understood to encompass functional antibody fragments thereof. The term also encompasses intact or full-length antibodies, including antibodies of any class or sub-class, including IgG and sub-classes thereof (IgG1, IgG2, IgG3, IgG4), IgM, IgE, IgA, and IgD.

An anti-CCR5 monoclonal antibody or antigen-binding portion thereof for use in the methods disclosed herein may be non-human (e.g., murine), chimeric, humanized, or human. Immunoglobulin structure and function are reviewed, for example, in Harlow et al., Eds., Antibodies: A Laboratory Manual, Chapter 14 (Cold Spring Harbor Laboratory, Cold Spring Harbor, 1988).

The terms “VL” and “VH” refer to the variable binding region from an antibody light chain and an antibody heavy chain, respectively. The variable binding regions comprise discrete, well-defined sub-regions known as “complementarity determining regions” (CDRs) and “framework regions” (FRs). The terms “complementarity determining region,” and “CDR,” are synonymous with “hypervariable region” or “HVR,” and refer to sequences of amino acids within antibody variable regions, which, in general, together confer the antigen specificity and/or binding affinity of the antibody, wherein consecutive CDRs (i.e., CDR1 and CDR2, CDR2 and CDR3) are separated from one another in primary amino acid sequence by a framework region. There are three CDRs in each variable region (HCDR1, HCDR2, HCDR3; LCDR1, LCDR2, LCDR3; also referred to as CDRHs and CDRLs, respectively). In certain embodiments, an antibody VH comprises four FRs and three CDRs as follows: FR1-HCDR1-FR2-HCDR2-FR3-HCDR3-FR4; and an antibody VL comprises four FRs and three CDRs as follows: FR1-LCDR1-FR2-LCDR2-FR3-LCDR3-FR4. In general, the VH and the VL together form the antigen-binding site through their respective CDRs.

Numbering of CDR and framework regions may be determined according to any known method or scheme, such as the Kabat, Chothia, EU, IMGT, and AHo numbering schemes (see, e.g., Kabat et al., “Sequences of Proteins of Immunological Interest, US Dept. Health and Human Services, Public Health Service National Institutes of Health, 1991, 5^{th} ed.; Chothia and Lesk, J. Mol. Biol. 196:901-917 (1987)); Lefranc et al., Dev. Comp. Immunol. 27:55, 2003; Honegger and Plückthun, J. Mol. Bio. 309:657-670 (2001)). Equivalent residue positions can be annotated and for different molecules to be compared using Antigen receptor Numbering And Receptor Classification (ANARCI) software tool (2016, Bioinformatics 15:298-300). Accordingly, identification of CDRs of an exemplary variable domain (VH or VL) sequence as provided herein according to one numbering scheme is not exclusive of an antibody comprising CDRs of the same variable domain as determined using a different numbering scheme.

In some embodiments, the present disclosure provides use of an anti-CCR5 antibody or antigen binding fragment thereof having a light chain variable region (VL) that is at least 70% identical to SEQ ID NO: 1, at least 75% identical to SEQ ID NO: 1, at least 80% identical to SEQ ID NO: 1, at least 85% identical to SEQ ID NO: 1, or at least 90% identical to SEQ ID NO: 1. In some embodiments, the present disclosure provides use of an anti-CCR5 antibody or antigen binding fragment thereof having a light chain variable antibody region that is 70%-100% identical to SEQ ID NO: 1, 75%-100% identical to SEQ ID NO: 1, 80%-100% identical to SEQ ID NO: 1, 85%-100% identical to SEQ ID NO: 1, 90%-100% identical to SEQ ID NO: 1 or 91%-100% identical to SEQ ID NO: 1.

In some embodiments, the present disclosure provides use of an anti-CCR5 antibody or antigen binding fragment thereof having a light chain variable region (VL) that is at least 70% identical to amino acids 20-131 of SEQ ID NO: 1, at least 75% identical to amino acids 20-131 of SEQ ID NO: 1, at least 80% identical to amino acids 20-131 of SEQ ID NO: 1, at least 85% identical to amino acids 20-131 of SEQ ID NO: 1, or at least 90% identical to amino acids 20-131 of SEQ ID NO: 1. In some embodiments, the present disclosure provides use of an anti-CCR5 antibody or antigen binding fragment thereof having a light chain variable antibody region that is 70%-100% identical to amino acids 20-131 of SEQ ID NO: 1, 75%-100% identical to amino acids 20-131 of SEQ ID NO: 1, 80%-100% identical to amino acids 20-131 of SEQ ID NO: 1, 85%-100% identical to amino acids 20-131 of SEQ ID NO: 1, 90%-100% identical to amino acids 20-131 of SEQ ID NO: 1 or 91%-100% identical to amino acids 20-131 of SEQ ID NO: 1.

In some embodiments, the present disclosure provides use of an anti-CCR5 antibody or antigen binding fragment thereof having a heavy chain variable region (VH) that is at least 70% identical to SEQ ID NO:3, at least 75% identical to SEQ ID NO:3, at least 80% identical to SEQ ID NO: 3, at least 85% identical to SEQ ID NO:3, or at least 90% identical to SEQ ID NO:3. In some embodiments the present disclosure provides use of an anti-CCR5 antibody or antigen binding fragment thereof having a heavy chain antibody variable region that is 70%-100% identical to SEQ ID NO: 3, 75%-100% identical to SEQ ID NO: 3, 80%-100% identical to SEQ ID NO: 3, 85%-100% identical to SEQ ID NO: 3, 90%-100% identical to SEQ ID NO: 3, or 91%-100% identical to SEQ ID NO:3.

In some embodiments, the present disclosure provides use of an anti-CCR5 antibody or antigen binding fragment thereof having a heavy chain variable region (VH) that is at least 70% identical to amino acids 20-141 of SEQ ID NO:3, at least 75% identical to amino acids 20-141 of SEQ ID NO:3, at least 80% identical to amino acids 20-141 of SEQ ID NO:3, at least 85% identical to amino acids 20-141 of SEQ ID NO:3, or at least 90% identical to amino acids 20-141 of SEQ ID NO: 3. In some embodiments the present disclosure provides use of an anti-CCR5 antibody or antigen binding fragment thereof having a heavy chain antibody variable region that is 70%-100% identical to amino acids 20-141 of SEQ ID NO: 3, 75%-100% identical to amino acids 20-141 of SEQ ID NO: 3, 80%-100% identical to amino acids 20-141 of SEQ ID NO: 3, 85%-100% identical to amino acids 20-141 of SEQ ID NO: 3, 90%-100% identical to amino acids 20-141 of SEQ ID NO: 3, or 91%-100% identical to amino acids 20-141 of SEQ ID NO:3.

In some embodiments, the present disclosure provides use of an anti-CCR5 antibody having a heavy chain variable region (VH) that is at least 70% identical to SEQ ID NO:5, at least 75% identical to SEQ ID NO: 5, at least 80% identical to SEQ ID NO: 5, at least 85% identical to SEQ ID NO: 5, or at least 90% identical to SEQ ID NO: 5. In some embodiments the present disclosure provides use of an anti-CCR5 antibody having a heavy chain variable antibody region that is 70%-100% identical to SEQ ID NO: 5, 75%-100% identical to SEQ ID NO: 5, 80%-100% identical to SEQ ID NO: 5, 85%-100% identical to SEQ ID NO: 5, 90%-100% identical to SEQ ID NO: 5, or 91%-100% identical to SEQ ID NO: 5.

In some embodiments, the present disclosure provides use of an anti-CCR5 antibody having a heavy chain variable region (VH) that is at least 70% identical to amino acids 20-141 of SEQ ID NO: 5, at least 75% identical to amino acids 20-141 of SEQ ID NO: 5, at least 80% identical to amino acids 20-141 of SEQ ID NO: 5, at least 85% identical to amino acids 20-141 of SEQ ID NO: 5, or at least 90% identical to amino acids 20-141 of SEQ ID NO: 5. In some embodiments the present disclosure provides use of an anti-CCR5 antibody having a heavy chain variable antibody region that is 70%-100% identical to amino acids 20-141 of SEQ ID NO: 5, 75%-100% identical to amino acids 20-141 of SEQ ID NO: 5, 80%-100% identical to amino acids 20-141 of SEQ ID NO: 5, 85%-100% identical to amino acids 20-141 of SEQ ID NO: 5, 90%-100% identical to amino acids 20-141 of SEQ ID NO: 5, or 91%-100% identical to amino acids 20-141 of SEQ ID NO: 5.

In some embodiments, the present disclosure provides use of an anti-CCR5 antibody or an antigen-binding fragment thereof comprising a heavy chain variable region (VH) and a light chain variable region (VL), wherein the VH comprises a heavy chain CDR1 (VH-CDR1) comprising the amino acid sequence of SEQ ID NO: 12, a heavy chain CDR2 (VH-CDR2) comprising the amino acid sequence of SEQ ID NO: 13, and a heavy chain CDR3 (VH-CDR3) comprising the amino acid sequence of SEQ ID NO: 14; and the VL comprises a light chain CDR1 (VL-CDR1) comprising the amino acid sequence of SEQ ID NO:9, a light chain CDR2 (VL-CDR2) comprising the amino acid sequence of SEQ ID NO: 10, and a light chain CDR3 (VL-CDR3) comprising the amino acid sequence of SEQ ID NO:11. In some such embodiments, the VH comprises an amino acid sequence that has at least 70%, 75%, 80%, 85%, 90%, 91%, 92%, 93%, 94%, 95%, 96%, 97%, 98%, or 99% identity with the amino acid sequence of SEQ ID NO:3 or amino acids 20-141 of SEQ ID NO:3, and a VL comprises an amino acid sequence that has at least 70%, 75%, 80%, 85%, 90%, 91%, 92%, 93%, 94%, 95%, 96%, 97%, 98%, or 99% identity with the amino acid sequence of SEQ ID NO: 1 or amino acids 20-131 of SEQ ID NO: 1, provided that the amino acid sequences of the VH-CDRs (SEQ ID NOS: 12-14) and VL-CDRs (SEQ ID NOS: 9-11) are unchanged; or the VH comprises an amino acid sequence that has at least 70%, 75%, 80%, 85%, 90%, 91%, 92%, 93%, 94%, 95%, 96%, 97%, 98%, or 99% identity with the amino acid sequence of SEQ ID NO:5 or amino acids 20-141 of SEQ ID NO:5, and a VL comprises an amino acid sequence that has at least 70%, 75%, 80%, 85%, 90%, 91%, 92%, 93%, 94%, 95%, 96%, 97%, 98%, or 99% identity with the amino acid sequence of SEQ ID NO: 1 or amino acids 20-131 of SEQ ID NO:1, provided that the amino acid sequences of the VH-CDRs (SEQ ID NOS: 12-14) and VL-CDRs (SEQ ID NOS: 9-11) are unchanged.

In some embodiments, the present disclosure provides use of an anti-CCR5 antibody or an antigen-binding fragment thereof comprising: (a) a VH comprising an amino acid sequence of SEQ ID NO: 3 or amino acids 20-141 of SEQ ID NO:3, and a VL comprising an amino acid sequence of SEQ ID NO: 1 or amino acids 20-131 of SEQ ID NO:1; or (b) a VH comprising an amino acid sequence of SEQ ID NO:5 or amino acids 20-141 of SEQ ID NO:5, and a VL comprising an amino acid sequence of SEQ ID NO: 1 or amino acids 20-131 of SEQ ID NO:1.

In some embodiments, the present disclosure provides use of an anti-CCR5 antibody comprising a heavy chain (HC) and a light chain (LC). The heavy chain typically comprises a VH and a heavy chain constant region (CH). Depending on the antibody isotype from which it derives, a heavy chain constant region may comprise CH1, CH2, and CH3 domains (IgA, IgD, IgG), or CH1, CH2, CH3, and CH4 domains (IgE, IgM). In some embodiments, the heavy chain constant region comprises a human IgG1, IgG2, IgG3, or lgG4 constant region. In some embodiments, the constant region of the anti-CCR5 antibody is an IgG4 constant region. The light chain typically comprises a VL and a light chain constant region (CL). In some embodiments, a CL comprises a C kappa (“CK”) constant region. In some embodiments, a CL comprises a C lambda (Cλ) constant region. In some embodiments, an anti-CCR5 antibody of the present disclosure comprises two heavy chains and two light chains, held together covalently by disulfide bridges.

In some embodiments, the present disclosure provides use of an anti-CCR5 antibody comprising a heavy chain constant region portion having an effect on antibody stability by way of, for example, one or more amino acid substitutions or deletions in the CH1 region known in the art. An amino acid modification (e.g., substitution) to modify (e.g., improve, reduce, or ablate) antibody stability includes, for example, S131C.

The S131C mutation promotes the formation of disulfide bonds linking antibody heavy and light chains, which, in turn, helps prevent dissociation of the heavy and light chain after administration. This dissociation phenomena is sometimes referred to a Fab exchange and occurs in IgG4 molecules. Details of Fab exchange and the S131C mutation have been discussed in Aran F. Labrijn, et. al. “Species-Specific Determinants in the IgG CH3 Domain Enable Fab-Arm Exchange by Affecting the Noncovalent CH3-CH3 Interaction Strength” J. Immunol. 15 Sep. 2011; 187 (6): 3238-3246.

In some embodiments, the present disclosure provides use of an anti-CCR5 antibody comprising a Fc region portion. As used herein, “Fc region portion” refers to the heavy chain constant region segment of the Fc fragment (the “fragment crystallizable” region or Fc region) from an antibody, which can include one or more constant domains, such as CH2, CH3, CH4 or any combination thereof. In some embodiments, an Fc region portion includes the CH2 and CH3 domains of an IgG, IgA, or IgD antibody or any combination thereof, or the CH3 and CH4 domains of an IgM or IgE antibody, and any combination thereof. In some embodiments, a CH2CH3 or a CH3CH4 structure has sub-region domains from the same antibody isotype and are human, such as human IgG1, IgG2, IgG3, IgG4, IgA1, IgA2, IgD, IgE, or IgM (e.g., CH2CH3 from human IgG1). By way of background, an Fc region is responsible for the effector functions of an antibody, such as ADCC (antibody-dependent cell-mediated cytotoxicity), CDC (complement-dependent cytotoxicity) and complement fixation, binding to Fc receptors (e.g., CD16, CD32, FcRn), greater half-life in vivo relative to a polypeptide lacking an Fc region, protein A binding, and perhaps even placental transfer (see Capon et al. Nature 337:525, 1989). In some embodiments, a Fc region portion in an antibody or antigen-binding fragment of the present disclosure is capable of mediating one or more of these effector functions. In some embodiments, a Fc region portion in an antibody or antigen-binding fragment of the present disclosure has normal effector function, meaning having less than 20%, 15%, 10%, 5%, 1% difference in effector function (e.g., ADCC, CDC, half-life or any combination thereof) as compared to a wild type IgG1 antibody.

In some embodiments, the present disclosure provides use of an anti-CCR5 antibody comprising a Fc region portion having an increase in one or more of these effector functions by way of, for example, one or more amino acid substitutions or deletions in the Fc region portion known in the art. An antibody or antigen-binding fragment having a mutated or variant Fc region portion having increased effector function means that the antibody exhibits an increase of at least 20%, 25%, 30%, 35%, 40%, 45%, 50%, 55%, 60%, 65%, 70%, 75%, 80%, 85%, 90%, 95%, or 99% in FcR binding, ADCC, CDC, or any combination thereof, as compared to an antibody having a wild type Fc region portion. In some embodiments, the mutated or variant Fc region portion exhibits increased binding to FcRn, FcγRI (CD64), FcγRIIA (CD32), FcγRIIIA (CD16a), FcγRIIIB (CD16b), or any combination thereof. In some embodiments, the Fc region portion in an antibody or antigen-binding fragment of the present disclosure is a variant Fc region portion having increased ADCC, CDC, half-life, or any combination thereof.

Amino acid modifications (e.g., substitutions) to modify (e.g., improve, reduce, or ablate) Fc functionalities include, for example, the M428L/N434S (“LS”) and L234A/L235A (“LALA”) mutations, which mutations are summarized and annotated in “Engineered Fc Regions”, published by InvivoGen (2011) and available online at www.invivogen.com/PDF/review/review-Engineered-Fc-Regions-invivogen.pdf?utm_source=review&utm_medium=pdf&utm_campaign=review&utm_content=Engineered-Fc-Regions, and are incorporated herein by reference.

In a preferred embodiment, the present disclosure provides use of an anti-CCR5 antibody comprising M428L/N434S, L234A/L235A, or S131C mutations, and any combinations thereof. These modification may be present in an human IgG Fc, in particular a human IgG1 or IgG4.

In some embodiments, the present disclosure provides use of an anti-CCR5 antibody that comprises a heavy chain (HC) and a light chain (LC), wherein the HC comprises an amino acid sequence that has at least 90%, 91%, 92%, 93%, 94%, 95%, 96%, 97%, 98%, 99%, or 100% identity with the amino acid sequence of SEQ ID NO:7, and the LC comprises an amino acid sequence that has at least 90%, 91%, 92%, 93%, 94%, 95%, 96%, 97%, 98%, or 99% identity with the amino acid sequence of SEQ ID NO:8

In some embodiments, the present disclosure provides use of an anti-CCR5 antibody that comprises a HC comprising an amino acid sequence that has the amino acid sequence of SEQ ID NO: 7, and a LC comprising an amino acid sequence that has the amino acid sequence of SEQ ID NO: 8.

As used herein, unless otherwise provided, a position of an amino acid residue in the constant region of human IgG1 heavy chain is numbered assuming that the variable region of human IgG1 is composed of 128 amino acid residues according to the Kabat numbering convention. The numbered constant region of human IgG1 heavy chain is then used as a reference for numbering amino acid residues in constant regions of other immunoglobulin heavy chains. A position of an amino acid residue of interest in a constant region of an immunoglobulin heavy chain other than human IgG1 heavy chain is the position of the amino acid residue in human IgG1 heavy chain with which the amino acid residue of interest aligns. Alignments between constant regions of human IgG1 heavy chain and other immunoglobulin heavy chains may be performed using software programs known in the art, such as the Megalign program (DNASTAR Inc.) using the Clustal W method with default parameters. According to the numbering system described herein, for example, although human IgG2 CH2 region may have an amino acid deletion near its amino-terminus compared with other C_{H2} regions, the position of the “N” located at 296 in human IgG2 C_{H2} is still considered position 297 because this residue aligns with “N” at position 297 in human IgG1 C_{H2}.

In some embodiments, the present disclosure provides use of leronlimab (PRO140) antibody or antigen binding fragment thereof. Leronlimab (PRO140) is a humanized IgG4 monoclonal antibody that binds to CCR5 described in U.S. Pat. Nos. 7,122,185 and 8,821,877, which are incorporated herein by reference, in their entirety. Leronlimab (PRO 140) is a humanized version of the murine monoclonal antibody, PA14, which was generated against CD4⁺ CCR5⁺ cells. Olson et al., Differential Inhibition of Human Immunodeficiency Virus Type 1 Fusion, gp 120 Binding and CC-Chemokine Activity of Monoclonal Antibodies to CCR5, J. VIROL., 73:4145-4155. (1999). PRO 140 binds to CCR5 expressed on the surface of a cell, and potently inhibits HIV-1 entry and replication at concentrations that do not affect CCR5 chemokine receptor activity in vitro and in the hu-PBL-SCID mouse model of HIV-1 infection. Olson et al., Differential Inhibition of Human Immunodeficiency Virus Type 1 Fusion, gp 120 Binding and CC-Chemokine Activity of Monoclonal Antibodies to CCR5, J. VIROL., 73:4145-4155. (1999); Trkola et al., Potent, Broad-Spectrum Inhibition of Human Immunodeficiency Virus Type 1 by the CCR5 Monoclonal Antibody PRO 140, J. VIROL., 75:579-588 (2001). As used herein, unless context clearly indicates otherwise, “leronlimab” refers to antibodies having CDRs matching those of leronlimab as described in U.S. Pat. Nos. 7,122,185 and 8,821,877 and which may include any a Fc as conventionally known or of the various Fc modifications described herein.

Leronlimab does not downregulate CCR5 surface expression or deplete CCR5-expressing cells, but does prevent CCL5-induced calcium mobilization in CCR5+ cells with an IC₅₀ of 45 μg/ml. In some embodiments, a CCR5 binding agent does not downregulate CCR5 surface expression, deplete CCR5-expressing cells, or both. In some embodiments, a CCR5 binding agent inhibits CCL5-induced calcium mobilization of CCR5+ cells with an IC₅₀ of 45 μg/ml. In some embodiments, the CCR5 binding agent is leronlimab.

Leronlimab (PRO 140) binds to CCR5 and blocks viral entry by interfering with the final phase of viral binding to the cell surface prior to fusion of the viral and cell membranes. Leronlimab (PRO 140) has been administered intravenously or subcutaneously to more than 750 healthy and HIV-1 infected individuals in Phase I/II/III studies. The drug has been well tolerated following intravenous administration of single doses of 0.5 to 10 mg/kg or up to 700 mg weekly doses as subcutaneous (SC) injection. Overall, 324 patients have been exposed to leronlimab (PRO 140) 350 mg SC weekly dose with the longest duration of exposure lasting 4 years. Similarly, more than 250 and 150 patients have been exposed to leronlimab (PRO 140) 525 mg and 700 mg SC weekly dose, respectively.

In some embodiments, the present disclosure provides use of an anti-CCR5 antibody that binds to the same epitope as that to which leronlimab binds or competes with leronlimab in binding to CCR5. Leronlimab binds to a discontinuous epitope spanning multiple extracellular domains on CCR5, which include the N-terminus and second extracellular loop (ECL2) of CCR5 (Trkola et al. J. Virol. 75:579-588, incorporated by reference in its entirety). Leronlimab directly blocks binding of HIV Env to the CCR5 co-receptor via a competitive mechanism. Leronlimab binding at least requires amino acid residues D2 in the N-terminus and R168 and Y176 in the ECL2; mutation of amino acids D95 and C101 in the ECL1, and C178 in ECL2 also affect leronlimab binding, e.g., by conformational perturbation (Olson et al. J. Virol. 73:4145-4155, incorporated by reference in its entirety). Targeted loss-of-function mutagenesis and subsequent photo-cross-linking using genetically encoded unnatural amino acids method was also used to map antibody-GPCR complexes and identified residues 174 and 175 at the amino-terminal end of ECL2 as forming the strongest links with leronlimab (Ray-Saha et al., Biochem. 53:1302-13010).

CCR5 amino acid residues that are involved in CCL5 (RANTES) binding include K1, D2, D11, E18, K26 in the N-terminus, D95 in the ECL1, and K171, K191, and R274 in the ECL2 (Navenot et al. J. Mol. Biol. 313:1181-1193, incorporated by reference in its entirety).

Nucleic acids encoding heavy and light chains of the humanized PA14 antibodies have been deposited with the ATCC. Specifically, the plasmids designated pVK-HuPRO140, pVg4-HuPRO140 (mut B+D+I) and pVg4-HuPRO140 HG2, respectively, were deposited pursuant to, and in satisfaction of, the requirements of the Budapest Treaty with the ATCC, Manassas, Va., U.S.A. 20108, on Feb. 22, 2002, under ATCC Accession Nos. PTA 4097, PTA 4099, and PTA 4098, respectively. The American Type Culture Collection (ATCC) is now located at 10801 University Boulevard, Manassas, Va. 20110-2209. The plasmids designated pVK-HuPRO140 and pVg4-HuPRO140 HG2 encode the light chain and heavy chain, respectively, of leronlimab.

The HCDR1-3 and LCDR1-3 amino acid sequences of leronlimab are set forth in SEQ ID NOS: 12-14 and 9-11, respectively. The heavy chain and light chain sequences of leronlimab are set forth in SEQ ID NOS: 7 and 8, respectively.

Leronlimab sequences with leader peptides, which are typically cleaved during final antibody processing are found in SEQ ID NOs: 19 and 20, with encoding nucleotide sequences in SEQ ID NOs: 21 and 22.

In some embodiments, the present disclosure provides use of a CCR5 binding agent that is a competitive inhibitor to CCR5. The term “competitive inhibitor” as used herein refers to a molecule that competes with a reference molecule for binding to a target, and thereby blunts, inhibits, dampens, reduces, or blocks the effects of the reference molecule on the target. Thus a competitive inhibitor to CCR5 would compete with CCL5 for binding to CCR5. In some embodiments, a competitive inhibitor to CCR5 is an antibody or antigen binding fragment thereof that comprises:
(i) a heavy chain variable region (VH) and a light chain variable region (VL), wherein the VH comprises a heavy chain CDR1 (VH-CDR1) comprising the amino acid sequence of SEQ ID NO: 12, a heavy chain CDR2 (VH-CDR2) comprising the amino acid sequence of SEQ ID NO:13, and a heavy chain CDR3 (VH-CDR3) comprising the amino acid sequence of SEQ ID NO:14; and the VL comprises a light chain CDR1 (VL-CDR1) comprising the amino acid sequence of SEQ ID NO: 9, a light chain CDR2 (VL-CDR2) comprising the amino acid sequence of SEQ ID NO:10, and a light chain CDR3 (VL-CDR3) comprising the amino acid sequence of SEQ ID NO:11;
(ii) a VH comprising the amino acid sequence of SEQ ID NO:3 or amino acids 20-141 of SEQ ID NO:3, and a VL comprising the amino acid sequence of SEQ ID NO: 1 or amino acids 20-131 of SEQ ID NO:1;

(iii) a VH comprises the amino acid sequence of SEQ ID NO:5 or amino acids 20-141 of SEQ ID NO:5, and a VL comprises the amino acid sequence of SEQ ID NO: 1 or amino acids 20-131 of SEQ ID NO: 1; or

(iv) a heavy chain comprising the amino acid sequence of SEQ ID NO:7 and a light chain comprising the amino acid sequence of SEQ ID NO:8.

In some embodiments, the present disclosure provides use of an anti-CCR5 antibody or an antigen-binding fragment thereof comprising: (a) a VH comprising an amino acid sequence of SEQ ID NO: 3 or amino acids 20-141 of SEQ ID NO:3, and a VL comprising an amino acid sequence of SEQ ID NO: 1 or amino acids 20-131 of SEQ ID NO:1; or (b) a VH comprising an amino acid sequence of SEQ ID NO:5 or amino acids 20-141 of SEQ ID NO:5, and a VL comprising an amino acid sequence of SEQ ID NO: 1 or amino acids 20-131 of SEQ ID NO:1.

In some embodiments, the present disclosure provides use of a CCR5 binding agent that is a competitive inhibitor to CCR5 and does not have CCL5 agonist activity upon binding to CCR5. CCL5 agonist activity may be detected by measuring a decrease in cAMP; induced cell migration; or both CAMP decrease and induced cell migration triggered in response to CCL5-CCR5 axis activity. Some CCR5 binding agents even while acting to inhibit, interrupt, block, mitigate, dampen, slow the progress of, or eliminate the triggering of the downstream effects of CCL5 on CCR5 receptor positive cells also gives rise to independent and separate CCL5 agonistic downstream CCL5/CCR5 axis signaling effects that may counteract or diminish the effectiveness of these CCR5 competitive inhibitors for the purposes of immunomodulatory regulation, alteration, or control for therapeutic purposes. In some embodiments, a competitive inhibitor to CCR5 that does not have CCL5 agonist activity is an antibody or antigen binding fragment thereof that comprises:
(i) a heavy chain variable region (VH) and a light chain variable region (VL), wherein the VH comprises a heavy chain CDR1 (VH-CDR1) comprising the amino acid sequence of SEQ ID NO: 12, a heavy chain CDR2 (VH-CDR2) comprising the amino acid sequence of SEQ ID NO:13, and a heavy chain CDR3 (VH-CDR3) comprising the amino acid sequence of SEQ ID NO:14; and the VL comprises a light chain CDR1 (VL-CDR1) comprising the amino acid sequence of SEQ ID NO: 9, a light chain CDR2 (VL-CDR2) comprising the amino acid sequence of SEQ ID NO:10, and a light chain CDR3 (VL-CDR3) comprising the amino acid sequence of SEQ ID NO:11;
(ii) a VH comprising the amino acid sequence of SEQ ID NO:3 or amino acids 20-141 of SEQ ID NO:3, and a VL comprising the amino acid sequence of SEQ ID NO:1 or amino acids 20-131 of SEQ ID NO:1;
(iii) a VH comprises the amino acid sequence of SEQ ID NO:5 or amino acids 20-141 of SEQ ID NO:5, and a VL comprises the amino acid sequence of SEQ ID NO: 1 or amino acids 20-131 of SEQ ID NO: 1; or
(iv) a heavy chain comprising the amino acid sequence of SEQ ID NO:7 and a light chain comprising the amino acid sequence of SEQ ID NO:8.

### Nucleic Acids, Vectors, and Host Cells

In another aspect, the present disclosure provides an isolated nucleic acid that encodes the anti-CCR5 antibody or antigen binding fragment thereof as described herein. In some embodiments, the isolated nucleic acid encodes the VH, the VL, or both the VH and VL of the antibody or antigen binding fragment thereof. In some embodiments, the isolated nucleic acid encodes the heavy chain, the light chain, or both the heavy and light chain of the antibody or antigen binding fragment thereof. In some embodiments, the nucleic acid encoding the anti-CCR5 antibody or antigen binding fragment thereof is codon optimized to enhance or maximize expression in certain types of cells (e.g., Scholten et al., Clin. Immunol. 119:135-145, 2006). As used herein a “codon optimized” polynucleotide is a heterologous polypeptide having codons modified with silent mutations corresponding to the abundances of host cell tRNA levels.

In some embodiments, a nucleic acid molecule encoding an anti-CCR5 antibody or antigen binding fragment thereof of the present disclosure (e.g., an antibody heavy chain and light chain, or VH and VL regions) comprises a nucleic acid sequence for a heavy chain or VH region and a light chain or VL, respectively, wherein the heavy chain or VH region is separated from the light chain or VL region by a 2A self-cleaving peptide. In some embodiments, the 2A self-cleaving peptide is a porcine teschovirus-1 (P2A), equine rhinitis A virus (E2A), Thosea asigna virus (T2A), foot-and-mouth disease virus (F2A), or any combination thereof (see, e.g., Kim et al., PLOS One 6: e18556, 2011, which 2A nucleic acid and amino acid sequences are incorporated herein by reference in their entirety).

In another aspect, an expression construct comprising a nucleic acid encoding an anti-CCR5 antibody or antigen binding fragment thereof as described herein is provided. In some embodiments, a nucleic acid may be operably linked to an expression control sequence (e.g., expression construct). As used herein, “expression construct” refers to a DNA construct containing a nucleic acid molecule that is operably-linked to a suitable control sequence capable of effecting the expression of the nucleic acid molecule in a suitable host. An expression construct may be present in a vector (e.g., a bacterial vector, a viral vector) or may be integrated into a genome. The term “operably linked” refers to the association of two or more nucleic acids on a single polynucleotide fragment so that the function of one is affected by the other. For example, a promoter is operably-linked with a coding sequence when it is capable of affecting the expression of that coding sequence (i.e., the coding sequence is under the transcriptional control of the promoter). The term “expression control sequence” (also called a regulatory sequence) refers to nucleic acid sequences that effect the expression and processing of coding sequences to which they are operably linked. For example, expression control sequences may include transcription initiation, termination, promoter and enhancer sequences; efficient RNA processing signals such as splicing and polyadenylation signals; sequences that stabilize cytoplasmic mRNA; sequences that enhance translation efficiency (i.e., Kozak consensus sequences); sequences that enhance protein stability; and possibly sequences that enhance protein secretion.

In some embodiments, a nucleic acid or an expression construct encoding an anti-CCR5 antibody or antigen binding fragment thereof is present in a vector. A “vector” is a nucleic acid molecule that is capable of transporting another nucleic acid. Vectors may be, for example, plasmids, cosmids, viruses, a RNA vector or a linear or circular DNA or RNA molecule that may include chromosomal, non-chromosomal, semi-synthetic or synthetic nucleic acids. Exemplary vectors are those capable of autonomous replication (episomal vector) or expression of nucleic acids to which they are linked (expression vectors). Exemplary viral vectors include retrovirus, adenovirus, parvovirus (e.g., adeno-associated viruses), coronavirus, negative strand RNA viruses such as ortho-myxovirus (e.g., influenza virus), rhabdovirus (e.g., rabies and vesicular stomatitis virus), paramyxovirus (e.g., measles and Sendai), positive strand RNA viruses such as picornavirus and alphavirus, and double-stranded DNA viruses including adenovirus, herpesvirus (e.g., Herpes Simplex virus types 1 and 2, Epstein-Barr virus, cytomegalovirus), and poxvirus (e.g., vaccinia, fowlpox and canarypox). Other viruses include Norwalk virus, togavirus, flavivirus, reoviruses, papovavirus, hepadnavirus, and hepatitis virus, for example. Examples of retroviruses include avian leukosis-sarcoma, mammalian C-type, B-type viruses, D type viruses, HTLV-BLV group, lentivirus, spumavirus (Coffin, J. M., Retroviridae: The viruses and their replication, In Fundamental Virology, Third Edition, B. N. Fields et al., Eds., Lippincott-Raven Publishers, Philadelphia, 1996). In some embodiments, a vector is a plasmid. In some other embodiments, a vector is a viral vector. In some such embodiments, the viral vector is a lentiviral vector or a γ-retroviral vector.

In a further aspect, the present disclosure also provides an isolated host cell comprising a nucleic acid, expression construct, or vector encoding an anti-CCR5 antibody or antigen binding fragment thereof as described herein. As used herein, the term “host” refers to a cell or microorganism targeted for genetic modification with a heterologous or exogenous nucleic acid molecule to produce a polypeptide of interest (e.g., an anti-CCR5 antibody or antigen-binding fragment thereof). In certain embodiments, a host cell may optionally already possess or be modified to include other genetic modifications that confer desired properties related or unrelated to biosynthesis of the heterologous or exogenous protein (e.g., inclusion of a selectable marker). More than one heterologous or exogenous nucleic acid molecule can be introduced into a host cell as separate nucleic acid molecules, as a plurality of individually controlled genes, as a polycistronic nucleic acid molecule, as a single nucleic acid molecule encoding a fusion protein, or any combination thereof. When two or more exogenous nucleic acid molecules are introduced into a host cell, it is understood that the two more exogenous nucleic acid molecules can be introduced as a single nucleic acid molecule (e.g., on a single vector), on separate vectors, integrated into the host chromosome at a single site or multiple sites. The number of referenced heterologous nucleic acid molecules or protein activities refers to the number of encoding nucleic acid molecules or the number of protein activities, not the number of separate nucleic acid molecules introduced into a host cell.

Examples of host cells include, but are not limited to, eukaryotic cells, e.g., yeast cells, animal cells, insect cells, plant cells; and prokaryotic cells, including E. coli. In some embodiments, the cells are mammalian cells. In some embodiments, the host cell is a human embryonic kidney (HEK293) cell, Y0 cell, Sp2/0 cell, NS0 murine myeloma cell, PER.C6® human cell, baby hamster kidney cell (BHK), COS cell, or Chinese hamster ovary (CHO) cell. In some embodiments, the host cell is a CHO-K1 cell. In some embodiments, the host cell is a CHOK1SV cell. Host cells are cultured using methods known in the art.

In yet another aspect, the present disclosure provides a process for making an anti-CCR5 antibody or antigen binding fragment thereof as described herein, comprising culturing a host cell of the present disclosure, under suitable conditions and for a sufficient time to express the anti-CCR5 antibody or antigen binding fragment thereof, and optionally isolating the anti-CCR5 antibody or antigen binding fragment thereof from the culture. Purification of soluble antibodies or antigen binding fragments thereof may be performed according to methods known in the art.

### Adenoviral-Associated Vectors

In another aspect, the vector used for delivering the nucleic acid or expression construct encoding an anti-CCR5 antibody or antigen binding fragment thereof is a recombinant adeno-associated virus vector (“rAAV”).

Adeno-associated virus (AAV) is a replication-deficient parvovirus, the single-stranded DNA genome of which is about 4.7 kb in length including 145 nucleotide inverted terminal repeat (ITRs). There are multiple serotypes of AAV. The nucleotide sequences of the genomes of the AAV serotypes are known. For example, the complete genome of AAV-1 is provided in GenBank Accession No. NC_002077; the complete genome of AAV-2 is provided in GenBank Accession No. NC_001401 and Srivastava et al., J. Virol., 45:555-564 (1983); the complete genome of AAV-3 is provided in GenBank Accession No. NC_1829; the complete genome of AAV-4 is provided in GenBank Accession No. NC_001829; the AAV-5 genome is provided in GenBank Accession No. AF085716; the complete genome of AAV-6 is provided in GenBank Accession No. NC_00 1862; at least portions of AAV-7 and AAV-8 genomes are provided in GenBank Accession Nos. AX753246 and AX753249, respectively (see also U.S. Pat. Nos. 7,282,199 and 7,790,449 relating to AAV-8); the AAV-9 genome is provided in Gao et al., J. Virol., 78: 6381-6388 (2004); the AAV-10 genome is provided in Mol. Ther., 13(1): 67-76 (2006); and the AAV-11 genome is provided in Virology, 330(2): 375-383 (2004). Cis-acting sequences directing viral DNA replication (rep), encapsidation/packaging and host cell chromosome integration are contained within the AAV ITRs. Three AAV promoters (named p5, p19, and p40 for their relative map locations) drive the expression of the two AAV internal open reading frames encoding rep and cap genes. The two rep promoters (p5 and p19), coupled with the differential splicing of the single AAV intron (at nucleotides 2107 and 2227), result in the production of four rep proteins (rep 78, rep 68, rep 52, and rep 40) from the rep gene. Rep proteins possess multiple enzymatic properties that are ultimately responsible for replicating the viral genome. The cap gene is expressed from the p40 promoter and it encodes the three capsid proteins VP1, VP2, and VP3. Alternative splicing and non-consensus translational start sites are responsible for the production of the three related capsid proteins. A single consensus polyadenylation site is located at map position 95 of the AAV genome. The life cycle and genetics of AAV are reviewed in Muzyczka, Current Topics in Microbiology and Immunology, 158: 97-129 (1992).

AAV possesses unique features that make it attractive as a vector for delivering foreign DNA to cells, for example, in gene therapy. AAV infection of cells in culture is noncytopathic, and natural infection of humans and other animals is silent and asymptomatic. Moreover, AAV infects many mammalian cells allowing the possibility of targeting many different tissues in vivo. Moreover, AAV transduces slowly dividing and non-dividing cells, and can persist essentially for the lifetime of those cells as a transcriptionally active nuclear episome (extrachromosomal element). The AAV proviral genome is infectious as cloned DNA in plasmids which makes construction of recombinant genomes feasible. Furthermore, because the signals directing AAV replication, genome encapsidation and integration are contained within the ITRs of the AAV genome, some or all of the internal approximately 4.3 kb of the genome (encoding replication and structural capsid proteins, rep-cap) may be replaced with foreign DNA. The rep and cap proteins may be provided in trans. Another significant feature of AAV is its stability. It easily withstands the conditions used to inactivate adenovirus (56° to 65° C. for several hours), making cold preservation of AAV less critical. AAV may also be lyophilized. Finally, AAV-infected cells are not resistant to superinfection.

In certain embodiments, AAV-based vectors provided herein comprise components from one or more serotypes of AAV. In certain embodiments, AAV based vectors provided herein comprise capsid components from one or more of AAV1, AAV2, AAV3, AAV4, AAV5, AAV6, AAV7, AAV8, AAV9, AAV10, AAV11, or AAVrh10. In preferred embodiments, AAV based vectors provided herein comprise components from the AAV9 serotype. As used herein, “AAV9 capsid” refers to the AAV9 having the amino acid sequence of GenBank accession: AAS99264, which is incorporated by reference herein. Some variation from this encoded sequence is encompassed by the present invention, which may include sequences having about 90%, 91%, 92%, 93%, 94%, 95%, 96%, 97%, 98%, 99% or 99.9% identical to the amino acid sequence of GenBank accession: AAS99264.

The recombinant adeno-associated virus (AAV) described herein may be generated using techniques which are known. See, e.g., WO 2003/042397; WO 2005/033321, WO 2006/110689; U.S. Pat. No. 7,588,772 B2. Such a method involves culturing a host cell which contains nucleic acid sequences encoding an AAV capsid; a functional rep gene; a expression cassette composed of, at a minimum, AAV inverted terminal repeats (ITRs) and a transgene; and sufficient helper functions to permit packaging of the expression cassette into the AAV capsid protein.

Briefly, cells are manufactured in a suitable cell culture (e.g., HEK 293) cells. Methods for manufacturing the gene therapy vectors described herein include methods well known in the art such as generation of plasmid DNA used for production of the gene therapy vectors, generation of the vectors, and purification of the vectors. In some embodiments, the gene therapy vector is an AAV vector and the plasmids generated are an AAV cis-plasmid encoding the AAV genome and the gene of interest, an AAV trans-plasmid containing AAV rep and cap genes, and an adenovirus helper plasmid. The vector generation process can include method steps such as initiation of cell culture, passage of cells, seeding of cells, transfection of cells with the plasmid DNA, post-transfection medium exchange to serum free medium, and the harvest of vector-containing cells and culture media. The harvested vector-containing cells and culture media are referred to herein as crude cell harvest.

The crude cell harvest may thereafter be subject method steps such as concentration of the vector harvest, diafiltration of the vector harvest, microfluidization of the vector harvest, nuclease digestion of the vector harvest, filtration of microfluidized intermediate, crude purification by chromatography, crude purification by ultracentrifugation, buffer exchange by tangential flow filtration, and/or formulation and filtration to prepare bulk vector.

A two-step affinity chromatography purification at high salt concentration followed anion exchange resin chromatography are used to purify the vector drug product and to remove empty capsids. These methods are described in more detail in U.S. Patent Application No. 62/322,071, filed Apr. 13, 2016 and U.S. Patent Application No. 62/226,357, filed Dec. 11, 2015 and entitled “Scalable Purification Method for AAV9”, which is incorporated by reference herein. Purification methods for AAV8 are U.S. Patent Application No. 62/322,098, filed Apr. 13, 2016 and U.S. Patent Application No. 62/266,341, filed Dec. 11, 2015, and rh10, U.S. Patent Application No. 62/322,055, filed Apr. 13, 2016 and U.S. Patent Application No. 62/266,347, entitled “Scalable Purification Method for AAVrh10”, filed Dec. 11, 2015, and for AAV1, U.S. Patent Application No. 62/322,083, filed Apr. 13, 2016 and US Patent Application No 62/26,351, for “Scalable Purification Method for AAV1”, filed Dec. 11, 2015, are all incorporated by reference herein.

### Pharmaceutical Compositions

In another aspect, the present disclosure provides use of pharmaceutical compositions comprising rAAVs for the in vivo delivery of CCR5 binding agents described herein for administration to a patient in need thereof. Pharmaceutical compositions can comprise the rAAVs described herein and one or more pharmaceutically acceptable carriers, diluents, or excipients, suitable for administration by a selected route. A pharmaceutical composition can comprise any rAAV described herein

Such excipients, carriers, diluents, and buffers include any pharmaceutical agent that can be administered without undue toxicity. Pharmaceutically acceptable excipients include, but are not limited to, liquids such as water, saline, glycerol and ethanol. Pharmaceutically acceptable salts can be included therein, for example, mineral acid salts such as hydrochlorides, hydrobromides, phosphates, sulfates, and the like; and the salts of organic acids such as acetates, propionates, malonates, benzoates, and the like. Additionally, auxiliary substances, such as wetting or emulsifying agents, pH buffering substances, and the like, may be present in such vehicles. A wide variety of pharmaceutically acceptable excipients are known in the art and need not be discussed in detail herein. Pharmaceutically acceptable excipients have been amply described in a variety of publications, including, for example, A. Gennaro, (2000) Remington: The Science and Practice of Pharmacy, 20th edition, Lippincott, Williams, & Wilkins; Pharmaceutical Dosage Forms and Drug Delivery Systems (1999) H. C. Ansel et al., eds., 7th ed., Lippincott, Williams, & Wilkins; and Handbook of Pharmaceutical Excipients (2000) A. H. Kibbe et al., eds., 3rd ed. Amer. Pharmaceutical Assoc.

A subject composition can comprise a liquid comprising a subject variant AAV capsid polypeptide of the invention or AAV virion comprising a variant AAV capsid polypeptide in solution, in suspension, or both. As used herein, liquid compositions include gels. In some cases, the liquid composition is aqueous. In some embodiments, the composition is an in situ gellable aqueous composition, e.g., an in situ gellable aqueous solution.

Such compositions include solvents (aqueous or non-aqueous), solutions (aqueous or non-aqueous), emulsions (e.g., oil-in-water or water-in-oil), suspensions, syrups, elixirs, dispersion and suspension media, coatings, isotonic and absorption promoting or delaying agents, compatible with pharmaceutical administration or in vivo contact or delivery. Aqueous and non-aqueous solvents, solutions and suspensions may include suspending agents and thickening agents. Such pharmaceutically acceptable carriers include tablets (coated or uncoated), capsules (hard or soft), microbeads, powder, granules and crystals. Supplementary active compounds (e.g., preservatives, antibacterial, antiviral and antifungal agents) can also be incorporated into the compositions.

Pharmaceutical compositions can be formulated to be compatible with a particular route of administration or delivery, as set forth herein or known to one of skill in the art. Thus, pharmaceutical compositions include carriers, diluents, or excipients suitable for administration by various routes.

The pharmaceutical compositions described herein can be formulated for oral, topical, transdermal, inhalation, parenteral, sublingual, buccal, rectal, vaginal, and intranasal administration. The term “parenteral”, as used herein, includes subcutaneous, intravenous, intramuscular, intrasternal, and intratumoral injection or infusion techniques. In some embodiments, the pharmaceutical compositions described herein are formulated for administration as an injection, e.g., an intravenous or subcutaneous injection. Non-limiting examples of formulations for injection can include a sterile suspension, solution or emulsion in oily or aqueous vehicles. Suitable oily vehicles can include, but are not limited to, lipophilic solvents or vehicles such as fatty oils or synthetic fatty acid esters, or liposomes. Aqueous injection suspensions can contain substances which increase the viscosity of the suspension. The suspension can also contain suitable stabilizers. Alternatively, the pharmaceutical compositions described herein can be lyophilized or in powder form for reconstitution with a suitable vehicle, e.g., sterile pyrogen-free water, before use. Compositions suitable for parenteral administration comprise aqueous and non-aqueous solutions, suspensions or emulsions of the active compound. Preparations are typically sterile and can be isotonic with the blood of the intended recipient. Non-limiting illustrative examples include water, saline, dextrose, fructose, ethanol, animal, vegetable or synthetic oils.

For transmucosal or transdermal administration (e.g., topical contact), penetrants can be included in the pharmaceutical composition. Penetrants are known in the art, and include, for example, for transmucosal administration, detergents, bile salts, and fusidic acid derivatives. For transdermal administration, the active ingredient can be formulated into aerosols, sprays, ointments, salves, gels, or creams as generally known in the art. For contact with skin, pharmaceutical compositions typically include ointments, creams, lotions, pastes, gels, sprays, aerosols, or oils. Useful carriers include Vaseline®, lanolin, polyethylene glycols, alcohols, transdermal enhancers, and combinations thereof.

Pharmaceutical compositions can include a suspension of the recombinant vector in a formulation buffer comprising a physiologically compatible aqueous buffer (e.g., in the range of pH 6 to 9, or pH 6.5 to 8, pH 7.0 to 7.7, or pH 7.2 to 7.8), a surfactant and optional excipients.

Cosolvents and adjuvants may be added to the formulation. Non-limiting examples of cosolvents contain hydroxyl groups or other polar groups, for example, alcohols, such as isopropyl alcohol; glycols, such as propylene glycol, polyethyleneglycol, polypropylene glycol, glycol ether; glycerol; polyoxyethylene alcohols and polyoxyethylene fatty acid esters. Adjuvants include, for example, surfactants such as, soya lecithin and oleic acid; sorbitan esters such as sorbitan trioleate; and polyvinylpyrrolidone.

Pharmaceutical compositions and delivery systems appropriate for the AAV vector or AAV virion and methods and uses of are known in the art (see, e.g., Remington: The Science and Practice of Pharmacy (2003) 20th ed., Mack Publishing Co., Easton, Pa.; Remington's Pharmaceutical Sciences (1990) 18th ed., Mack Publishing Co., Easton, Pa.; The Merck Index (1996) 12th ed., Merck Publishing Group, Whitehouse, N.J.; Pharmaceutical Principles of Solid Dosage Forms (1993), Technonic Publishing Co., Inc., Lancaster, Pa.; Ansel and Stoklosa, Pharmaceutical Calculations (2001) 11th ed., Lippincott Williams & Wilkins, Baltimore, Md.; and Poznansky et al., Drug Delivery Systems (1980), R. L. Juliano, ed., Oxford, N. Y., pp. 253-315).

Doses can vary and depend upon whether the treatment is prophylactic or therapeutic, the type, onset, progression, severity, frequency, duration, or probability of the disease treatment is directed to, the clinical endpoint desired, previous or simultaneous treatments, the general health, age, gender, race or immunological competency of the subject and other factors that will be appreciated by the skilled artisan. The dose amount, number, frequency or duration may be proportionally increased or reduced, as indicated by any adverse side effects, complications or other risk factors of the treatment or therapy and the status of the subject. The skilled artisan will appreciate the factors that may influence the dosage and timing required to provide an amount sufficient for providing a therapeutic or prophylactic benefit.

The AAV vector can be formulated for administration in a unit dosage form in association with a pharmaceutically acceptable vehicle. Such vehicles can be inherently nontoxic, and non-therapeutic. A vehicle can be water, saline, Ringer's solution, dextrose solution, and 5% human serum albumin. Non-aqueous vehicles such as fixed oils and ethyl oleate can also be used. The vehicle can contain minor amounts of additives such as substances that enhance isotonicity and chemical stability (e.g., buffers and preservatives).

In some embodiments, the aqueous formulation comprises at least one lyoprotectant. In some such embodiments, the at least one lyoprotectant is selected from sucrose, arginine, glycine, sorbitol, glycerol, trehalose, dextrose, alpha-cyclodextrin, hydroxypropyl beta-cyclodextrin, hydroxypropyl gamma-cyclodextrin, proline, methionine, albumin, mannitol, maltose, dextran, and combinations thereof. In some embodiments, the lyoprotectant is sucrose. In some embodiments, the total concentration of lyoprotectant in the aqueous formulation is 3-12%, such as 5-12%, 6-10%, 5-9%, 7-9%, or 8%.

In some embodiments, the aqueous formulation comprises at least one surfactant. Exemplary surfactants include polysorbate 80, polysorbate 20, poloxamer 88, and combinations thereof. In some embodiments, the aqueous formulation comprises polysorbate 80. In some embodiments, the total concentration of the at least one surfactant is 0.01%-0.1%, such as 0.01%-0.05%, 0.01%-0.08%, or 0.01%-0.06%, 0.01%-0.04%, 0.01%-0.03%, or 0.02%.

In some embodiments, pharmaceutical compositions of the present invention are formulated in a single dose unit or in a form comprising a plurality of dosage units. Methods of preparing such dosage forms are known, or will be apparent, to those skilled in this art; for example, see Remington: The Science and Practice of Pharmacy, 20th Edition (Philadelphia College of Pharmacy and Science, 2000).

In some embodiments, the rAAV dose is 0.1-40×10¹² AAV genomes/kg, such as 0.1-40×10¹² AAV genomes/kg, 0.1-35×10¹² AAV genomes/kg, 0.1-30×10¹² AAV genomes/kg, 0.1-25×10¹² AAV genomes/kg, 0.1-15×10¹² AAV genomes/kg, 0.1-10×10¹² AAV genomes/kg, 0.1-5×10¹² AAV genomes/kg, 0.5-40×10¹² AAV genomes/kg, 0.5-35×10¹² AAV genomes/kg, 0.5-30×10¹² AAV genomes/kg, 0.5-25×10¹² AAV genomes/kg, 0.5-15×10¹² AAV genomes/kg, 0.5-10×10¹² AAV genomes/kg, 0.5-5×10¹² AAV genomes/kg, 1.0-40×10¹² AAV genomes/kg, 1.0-35×10¹² AAV genomes/kg, 1.0-30×10¹² AAV genomes/kg, 1.0-25×10¹² AAV genomes/kg, 1.0-15×10¹² AAV genomes/kg, 1.0-10×10¹² AAV genomes/kg, 1.0-5×10¹² AAV genomes/kg, 1.5-40×10¹² AAV genomes/kg, 1.5-35×10¹² AAV genomes/kg, 1.5-30×10¹² AAV genomes/kg, 1.5-25×10¹² AAV genomes/kg, 1.5-15×10¹² AAV genomes/kg, 1.5-10×10¹² AAV genomes/kg, or 1.5-5×10¹² AAV genomes/kg. In some embodiments, the rAAV dose is 1-4×10¹² AAV genomes/kg. In some embodiments, the rAAV dose is 2×10¹² AAV genomes/kg.

In any of the aforementioned pharmaceutical compositions, the CCR5 binding agent may be leronlimab, a modified leronlimab, or a fragment thereof.

### Methods of Use

The present disclosure provides methods of treating a patient at risk of contracting HIV infection or a patient having an HIV infection by administering to the patient an effective amount of a recombinant AAV expressing a CCR5 binding agent. In embodiments, the CCR5 binding agent comprises a leronlimab comprising one or more amino acid substitutions conferring increased effector function and circulation half-life, e.g. comprising M428L/N434S, L234A/L235A, and S131C mutations. Results of in vivo studies in rhesus macaques demonstrated that a recombinant rAAV expressing a CCR5 binding agent with modifications to enhance effector function and circulation half-life can suppress CCR5-tropic retroviral replication in chronic simian HIV infection.

Patients or patients that can be treated by AAV vectors of the present disclosure include, but are not limited to, a mammal, such as human or non-human primates (e.g., monkeys and apes). In embodiments, the patient is human. The patient can be male or female and can be any suitable age, including infant, juvenile, adolescent, adult, and geriatric. In embodiments, the patient is >65 years. In some embodiments, the patient is ≤65 years. In embodiments, the patient is ≥18 years and ≤65 years.

Patients may also be administered a AAV vector in anticipation of exposure to HIV, upon exposure to HIV, at the initial detection of infection with HIV, upon the development of new symptoms after infection, or during any period of HIV infection, including AIDS.

Subject variant AAV capsid polypeptides or subject AAV vectors can be administered systemically, regionally or locally, or by any route, for example, by injection, infusion, orally (e.g., ingestion or inhalation), or topically (e.g., transdermally). Such delivery and administration methods include intravenously, intramuscularly, intraperitoneally, intradermally, subcutaneously, intracavity, intracranially, transdermally (topical), parenterally, e.g. transmucosally or rectally. Exemplary administration and delivery routes include intravenous, intraperitoneal, intraarterial, intramuscular, parenteral, subcutaneous, intra-pleural, topical, dermal, intradermal, transdermal, parenterally, e.g. transmucosal, intra-cranial, intra-spinal, oral (alimentary), mucosal, respiration, intranasal, intubation, intrapulmonary, intrapulmonary instillation, buccal, sublingual, intravascular, intrathecal, intracavity, iontophoretic, intraocular, ophthalmic, optical, intraglandular, intraorgan, and intralymphatic.

Administration may be once or may continue for a set period of time, such as 1 week, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, 6 months, 9 months, 1 year, 2 years, or until at least HIV viral RNA is no longer detectable.

An appropriate dose, suitable duration, and frequency of administration of the AAV vector will be determined by such factors as the condition of the patient, size, weight, body surface area, age, sex, type and severity of the disease, particular therapy to be administered, particular form of the active ingredient, time and the method of administration, and other drugs being administered concurrently, which can readily be determined by a person skilled in the art.

Dosages can range from 0.1-40×10¹² AAV genomes/kg. Based upon the composition, the dose can be delivered at periodic intervals, e.g., on one or more separate occasions. Desired time intervals of multiple doses of a particular composition can be determined without undue experimentation by one skilled in the art.

In some embodiments, the AAV vector is administered to the patient once and each administration delivers from 0.1×10¹² genomes/kg to 40×10¹² genomes/kg of the AAV vector to the patient. In another embodiment, each administration delivers from 0.5×10¹² genomes/kg to 20×10¹² genomes/kg of the AAV vector to the patient. In another embodiment, each administration delivers from 1×10¹² genomes/kg to 10×10¹² genomes/kg of the AAV vector to the patient. In another embodiment, each administration delivers from 1×10¹² genomes/kg to 5×10¹² genomes/kg of the AAV vector to the patient. In another embodiment, each administration delivers 1×10¹² genomes/kg AAV vector to the patient. In another embodiment, each administration delivers 1.5×10¹² genomes/kg AAV vector to the patient. In another embodiment, each administration delivers 2×10¹² genomes/kg AAV vector to the patient. In another embodiment, each administration delivers 2.5×10¹² genomes/kg AAV vector to the patient. In another embodiment, each administration delivers 3×10¹² genomes/kg AAV vector to the patient. In another embodiment, each administration delivers 3.5×10¹² genomes/kg AAV vector to the patient. In another embodiment, each administration delivers 4×10¹² genomes/kg AAV vector to the patient.

In a preferred embodiment, the AAV vector delivers an anti-CCR5 antibody (e.g. leronlimab) comprising M428L/N434S, L234A/L235A, and S131C mutations.

In one embodiment, the AAV vector is administered once.

In one embodiment, the AAV vector is administered twice.

In one embodiment, the AAV vector is administered a plurality of times, and a first administration is separated from the subsequent administration by an interval of less than one week. In another embodiment, the first administration is separated from the subsequent administration by an interval of at least one week. In a further embodiment, the first administration is separated from the subsequent administration by an interval of one week. In another embodiment, the first administration is separated from the subsequent administration by an interval of two to four weeks. In another embodiment, the first administration is separated from the subsequent administration by an interval of two weeks. In a further embodiment, the first administration is separated from the subsequent administration by an interval of four weeks. In yet another embodiment, the AAV vector is administered a plurality of times, and a first administration is separated from the subsequent administration by an interval of at least one month. In yet another embodiment, the AAV vector is administered once a week for two weeks. In yet another embodiment, the AAV vector is administered once a week for four weeks. In yet another embodiment, the AAV vector is administered once per week as long as needed.

### EXAMPLES

### Example 1: Construction of an Adeno-Associated Virus (AAV) Expression Cassette for In Vivo Delivery of a Long-Acting Leronlimab

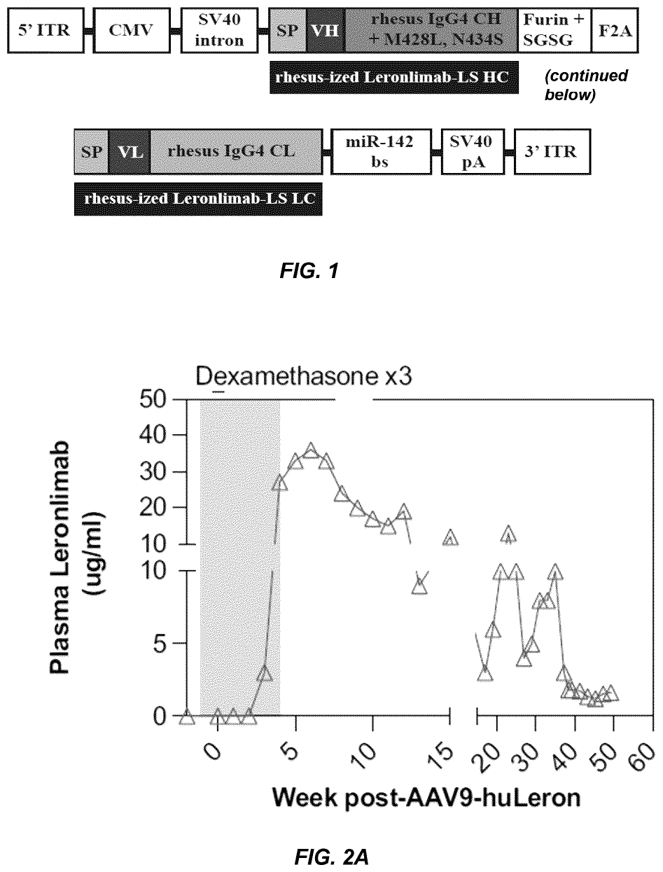

An adeno-associated virus (AAV) expression cassette was designed for in vivo delivery of a long-acting leronlimab. To package the parental leronlimab sequence and the MacLS (Macaque IgG4 with an LS mutation) sequence into AAV for further study, a previously described single-stranded AAV expression cassette that has successfully been used to express authentic IgG for long periods in macaques (FIG. 1; SEQ ID NO: 16) was used. In the construct, the Furin cleavage site with SGSG linker and F2A self-cleaving peptide separates the adjacent heavy and light chains to yield fully functional antibodies with no unnecessary additional amino acids. To prevent off-target expression in antigen presenting cells that may lead to development of antidrug antibodies (ADA), binding site sequences were inserted for microRNA (miR) miR-142-3p, a highly expressed myeloid-specific miR that prevents unwanted expression of genes in myeloid cells in vivo. The construct yields a self-cleaving, glycosylated, full-length authentic IgG molecule, as indicated by the black boxes in FIG. 1. The same construct was used to express human leronlimab.

### Example 2: In Vivo Efficacy of AAV-Expressed Leronlimab to Control Hiv Replication

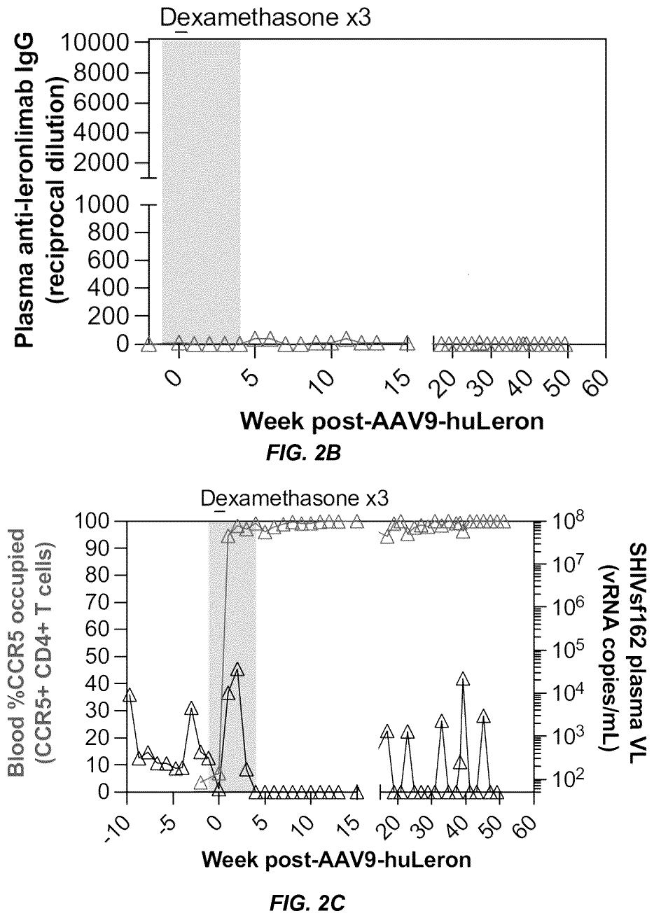

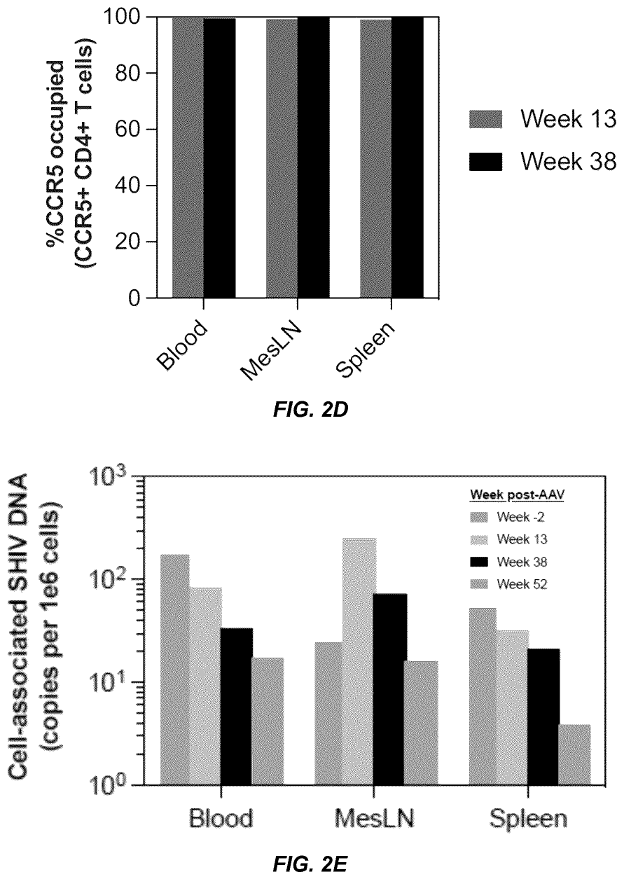

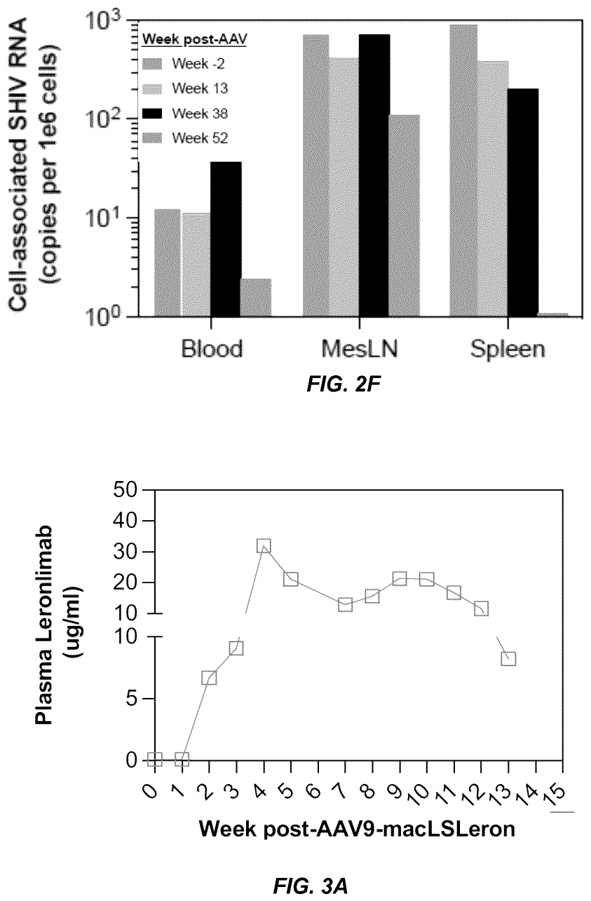

The ability of the leronlimab-delivering AAV to transduce myocytes, trigger them to produce leronlimab, and subsequently contain CCR5-tropic retroviral replication was tested. An AAV9 encoding human Fc leronlimab (AAV9-huLeron) was administered at 2×10¹² AAV genomes/kg of to a Mauritian cynomolgus macaque (MCM) chronically infected with the CCR5-tropic virus SHIV_{SF162P3}. To limit immune activation and prevent AAV clearance, dexamethasone (at −12, −1, and 5 hours post-AAV) and tacrolimus (daily, days −8 to 28 post-AAV) were administered. Leronlimab was detectable in plasma at week 3 after administration of leronlimab-AAV following achievement of full CCR5 receptor occupancy in peripheral blood (FIGS. 2A and 2C). Leronlimab persisted in the plasma for one year post leronlimab-AAV administration (FIG. 2A) without appearance of any ADA (FIG. 2B). Following leronlimab-AAV administration and full CCR5 RO on peripheral blood CD4+ T cells, plasma viral loads fell to undetectable levels. Plasma viremia “blips” that coincided with transient dips in CCR5 RO and plasma leronlimab levels were observed. Finally, CD4+ T cells from mesenteric lymph nodes (LN) and spleen maintained near full CCR5 RO (FIG. 2D). Cell-associated SHIV DNA (FIG. 2E) and RNA (FIG. 2F) decreased in blood and spleen, with minimal decreases in mesenteric LN.

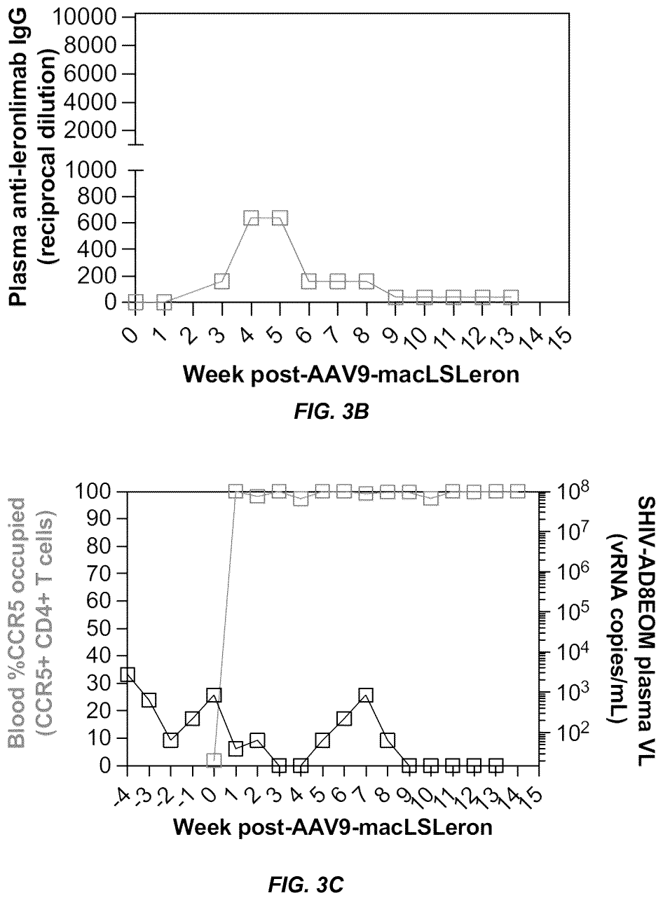

The in vivo efficacy of AAV-expressed leronlimab to control HIV replication and mediate a functional cure was tested in an in vivo rhesus macaque model of chronic simian HIV (SHIV) infection. An in vivo study was conducted, as described above, but with three changes: (1) immunosuppression around AAV injection was not included, (2) the macaque IgG4 LS variant, AAV9-macLSLeron, was used instead of human leronlimab, and (3) the SHIV_{AD8EO} strain was used for infection instead of SHIV_{SF162P3}. Full CCR5 RO on peripheral blood CD4+T cells was observed prior to the emergence of free leronlimab in plasma (FIGS. 3A and 3C). A transient disappearance of plasma viremia was observed, which was followed by rebound and subsequent full suppression of plasma viremia. The brief resurgence of plasma viremia was accompanied by ADA that was subsequently resolved along with plasma viremia (FIG. 3B).

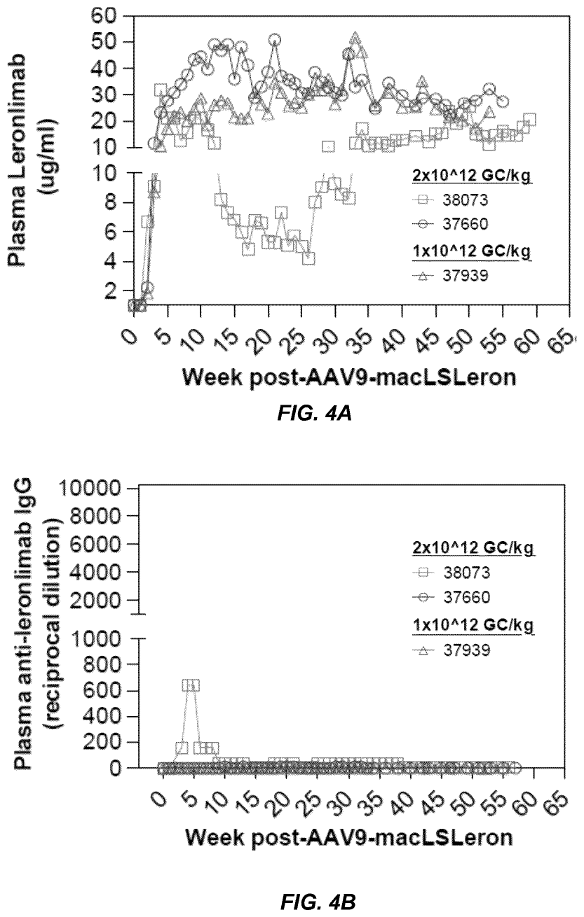

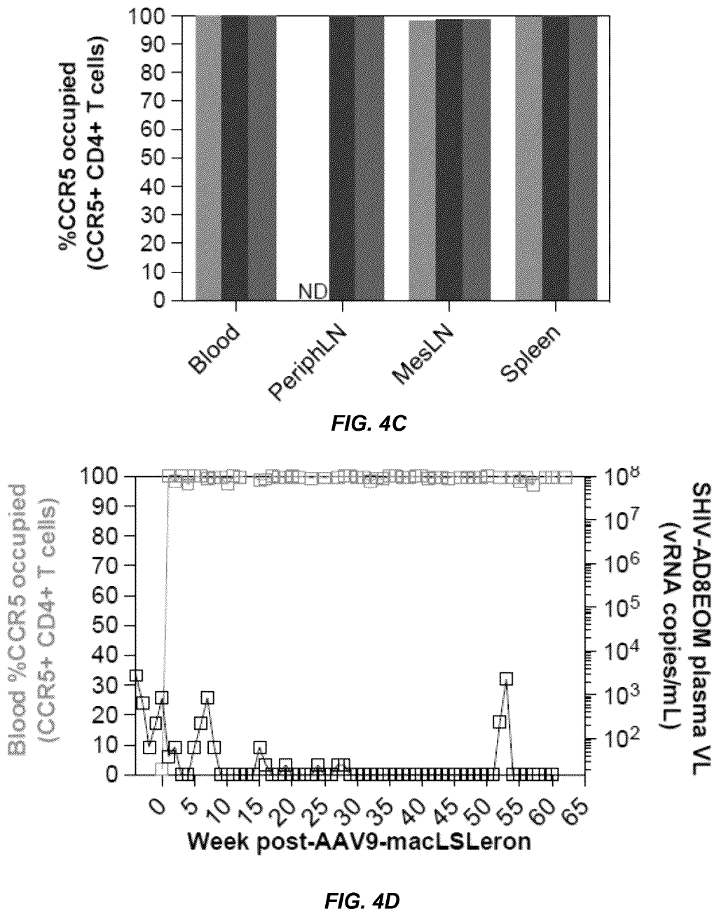

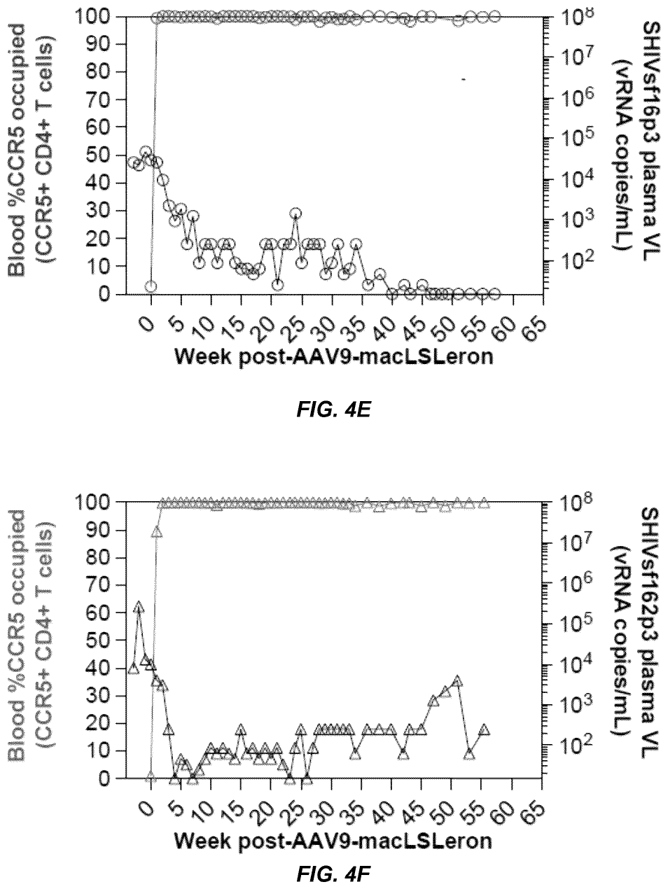

A similar study was performed using three SHIV_{AD8EOM}-infected rhesus macaques, two of which (designated 38073 and 37660) received 2×10¹² genomes/kg of AAV9-macLS, and one of which received only 1×10¹² genomes/kg of AAV9-macLS. As in the above single-macaque study, full CCR5 receptor occupancy was detected on peripheral blood CD4+ T cells prior to the emergence of free leronlimab in plasma (FIG. 4A, FIG. 4D, FIG. 4E, and FIG. 4F. In all three macaques, over a year of persistent leronlimab in plasma was observed with low to undetectable plasma viremia and minimal to no ADA. Furthermore, full and stable CCR5 receptor occupancy was been maintained on peripheral blood CD4+ T cells as well as on CD4+ T cells within peripheral LN, mesenteric LN, and spleen measured at weeks 23-26 post-AAV administration (FIG. 4C).

These in vivo studies demonstrate delivery of leronlimab by AAV and as a means to contain retroviral replication.

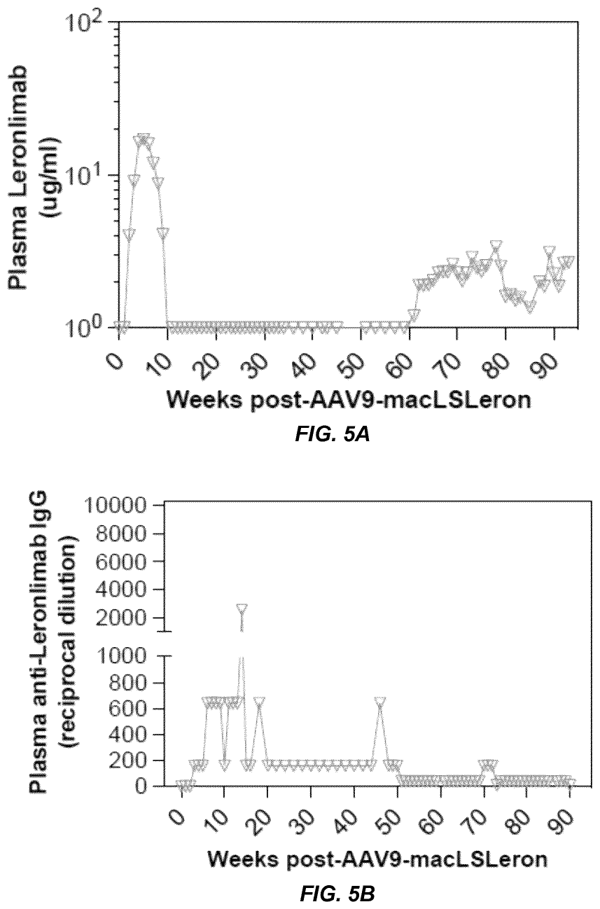

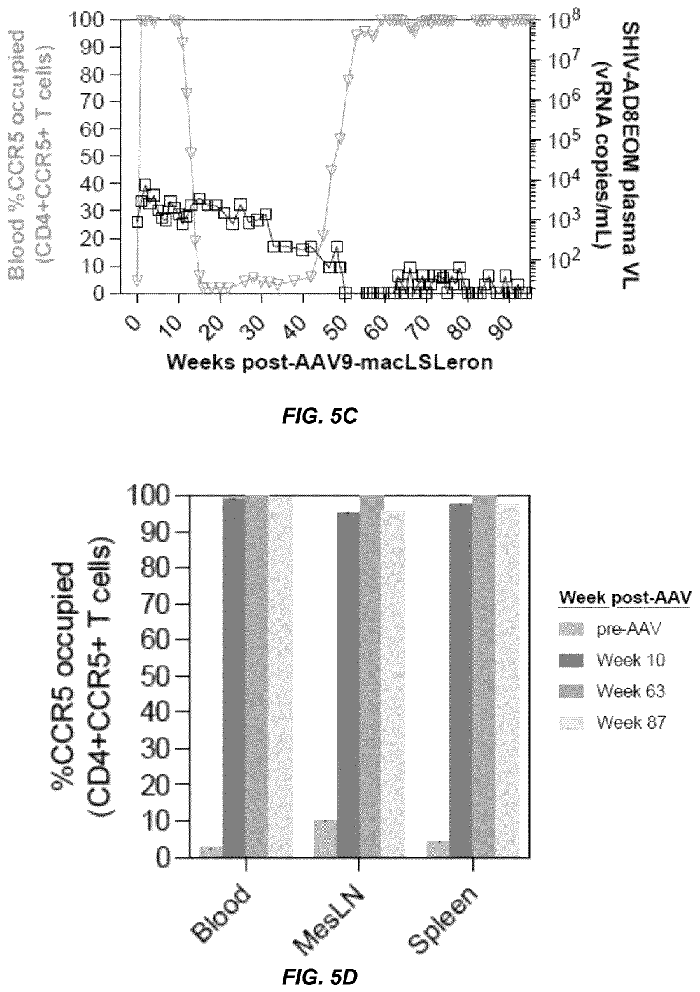

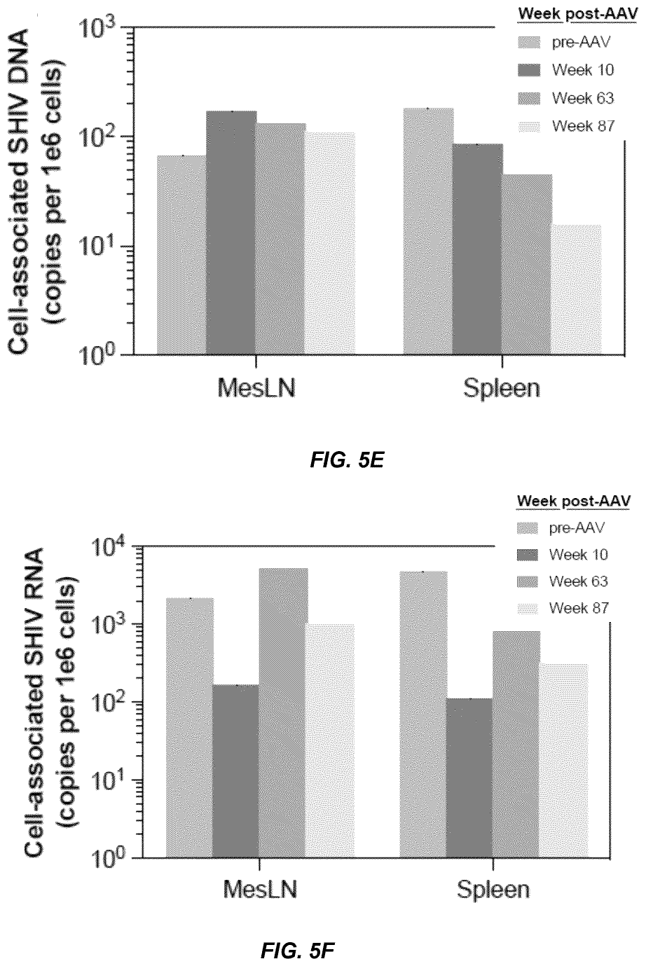

In addition to the long-term viral suppression mediated by AAV-expressed Leronlimab, a case of Leronlimab reexpression was observed after nearly six months of undetectable plasma Leronlimab and fully washed out CCR5 on blood CD4+ T cells (FIG. 5). One year following AAV administration (2×1012 AAV genomes/kg), plasma leronlimab increased in plasma once again, with minimal ADA development (FIG. 5A and FIG. 5B). Alongside increased plasma leronlimab, CCR5 receptor occupancy reached 100% and remarkably, plasma viral loads fell to undetectable levels with transient blips of plasma viremia (FIG. 5C). During this period of reexpression, CD4+ T cells in mesenteric lymph node and spleen maintained near 100% CCR5 receptor occupancy (FIG. 5D) with no effect on cell-associated SHIV DNA (FIG. 5E) or RNA (FIG. 5F) in mesenteric LN and approximately a log decrease in viral DNA and RNA in spleen.

SEQUENCE LISTING
>VL protein sequence; signal peptide at amino acids 1-19; CDRs underlined
SEQ ID NO: 1
MKLPVRLLVLMFWIPASSSDIVMTQSPLSLPVTPGEPASISCRSSQRLLSSYGHTYLHWYLQKPGQSPQLLIYEVS
NRFSGVPDRFSGSGSGTDFTLKISRVEAEDVGVYYCSQSTHVPLTFGQGTKVEIK
>VL nucleotide sequence
SEQ ID NO: 2
tctagaccaccatgaagttgcctgttaggctgttggtgctgatgttctggattcctgcttccagcagtgatattgt
gatgacccaatctccactctccctgcctgtcactcctggagagccagcctccatctcttgcagatctagtcagcgc
cttctgagcagttatggacatacctatttacattggtacctacagaagccaggccagtctccacagctcctgatct
acgaagtttccaaccgattttctggggtcccagacaggttcagtggcagtgggtcagggacagatttcacacttaa
gatcagtagagtggaggctgaggatgtgggagtttattactgctctcaa
agtacacatgttcctctcacgttcggacaggggaccaaggtggaaataaaacgtaagtagtcttctc
aactctaga
>PRO#2 VH protein sequence; signal peptide at amino acids 1-19;
CDRs underlined
SEQ ID NO: 3
MEWSGVFIFLLSVTAGVHSEVQLVESGGGLVKPGGSLRLSCAASGYTFSNYWIGWVRQAPGKGLEWIGDIYPG
GNYIRNNEKFKDKTTLSADTSKNTAYLQMNSLKTEDTAVYYCGSSFGSNYVFAWFTYWGQGTLVTVSS
>PRO#2 VH nucleotide sequence
SEQ ID NO: 4
acgcgtccaccatggaatggagcggagtctttatctttctcctgtcagtaactgcaggtgtccactccgaggtgca
gctggtggagtctggtggaggcttggtaaagcctggaggttcccttagactctcctgtgcagcctctggttacact
ttcagtaactattggatcggatgggtccgccaggctccaggcaaagggctggagtggattggcgatatctaccctg
gagggaactacatcaggaacaatgagaagttcaaggacaagaccaccctgtcagcagatacttccaagaacacagc
ctatctgcaaatgaacagcctgaaaaccgaggacacagccgtgtattactgtggaagcagcttcggtagtaactac
gtgttcgcctggtttacttactggggccaagggactctggtcacagtctcctcaggtgagtccttaaaacctctag
a
>PRO#1 VH protein sequence; signal peptide at amino acids 1-19;
CDRs underlined
SEQ ID NO: 5
MEWSGVFIFLLSVTAGVHSQVQLVQSGPDVKKPGTSMKMSCKTSGYTFSNYWIGWVRQAPGQGLEWIGDIYP
GGNYIRNNEKFKDKTTLTADTSTSTAYMQLGSLRSEDTAVYYCGSSFGSNYVFAWFTYWGQGTLVTVSS
>PRO#1 VH nucleotide sequence
SEQ ID NO: 6
tctagaccaccatggaatggagcggggtctttatctttctcctgtcagtaactgcaggtgtccactcccaggtcca
actggtgcagtctggacctgatgtgaaaaagcctgggacttcaatgaagatgtcctgcaagacgtctggatacacc
ttcagtaactattggatcggatgggttaggcaggcgcctggacaaggccttgagtggattggagatatttaccctg
gagggaactatatcaggaacaatgagaagttcaaggacaagaccacactgacggcagacacatcgaccagcacggc
ctacatgcaacttggcagcctgagatctgaagacactgccgtctattactgtggaagcagcttcggtagtaactac
gtgttcgcctggtttacttactggggccaagggactctggtcacagtctcctcaggtgagtccttaaaacctctag
a
>macaque IgG4 LS leronlimab heavy chain protein sequence (CDRs underlined)
SEQ ID NO: 7
EVQLVESGGGLVKPGGSLRLSCAASGYTFSNYWIGWVRQAPGKGLEWIGDIYPGGNYIRNNEKFKDKTTLSAD
TSKNTAYLQMNSLKTEDTAVYYCGSSFGSNYVFAWFTYWGQGTLVTVSSASTKGPSVFPLASCSRSTSESTAA
LGCLVKDYFPEPVTVSWNSGALTSGVHTFPAVLQSSGLYSLSSVVTVPSSSLGTQTYVCNVVHEPSNTKVDKRV
EFTPPCPPCPAPEAAGGPSVFLFPPKPKDTLMISRTPEVTCVVVDVSQEDPEVQFNWYVDGVEVHNAQTKPRER
QFNSTYRVVSVLTVTHQDWLNGKEYTCKVSNKGLPAPIEKTISKAKGQPREPQVYILPPPQEELTKNQVSLTCL
VTGFYPSDIAVEWESNGQPENTYKTTPPVLDSDGSYLLYSKLTVNKSRWQPGNIFTCSVLHEALHSHYTQKSLS
VSPGK
>macaque IgG4 LS leronlimab light chain protein sequence (CDRs underlined)
SEQ ID NO: 8
DIVMTQSPLSLPVTPGEPASISCRSSQRLLSSYGHTYLHWYLQKPGQSPQLLIYEVSNRFSGVPDRFSGSGSGTDF
TLKISRVEAEDVGVYYCSQSTHVPLTFGQGTKVEIKRAVAAPSVFIFPPSEDQVKSGTVSVVCLLNNFYPREASV
KWKVDGVLKTGNSQESVTEQDSKDNTYSLSSTLTLSSTDYQSHNVYACEVTHQGLSSPVTKSFNRGEC
>LCDR1 amino acid sequence
SEQ ID NO: 9
RSSQRLLSSYGHTYLH
>LCDR2 amino acid sequence
SEQ ID NO: 10
EVSNRFS
>LCDR3 amino acid sequence
SEQ ID NO: 11
SQSTHVPLT
>HCDR1 amino acid sequence
SEQ ID NO: 12
NYWIG
HCDR2 amino acid sequence
SEQ ID NO: 13>
DIYPGGNYIRNNEKFKD
>HCDR3 amino acid sequence
SEQ ID NO: 14
SFGSNYVFAWFTY
>Homo sapiens CCR5, NCBI Reference Sequence: NP_000570.1
SEQ ID NO: 15
MDYQVSSPIYDINYYTSEPCQKINVKQIAARLLPPLYSLVFIFGFVGNMLVILILINCKRLKSMTDIYLLNLAISDL
FFLLTVPFWAHYAAAQWDFGNTMCQLLTGLYFIGFFSGIFFIILLTIDRYLAVVHAVFALKARTVTFGVVTSVIT
WVVAVFASLPGIIFTRSQKEGLHYTCSSHFPYSQYQFWKNFQTLKIVILGLVLPLLVMVICYSGILKTLLRCRNE
KKRHRAVRLIFTIMIVYFLFWAPYNIVLLLNTFQEFFGLNNCSSSNRLDQAMQVTETLGMTHCCINPIIYAFVGE
KFRNYLLVFFQKHIAKRFCKCCSIFQQEAPERASSVYTRSTGEQEISVGL
>macaque IgG4 LS leronlimab AAV expression cassette
SEQ ID NO: 16
CAGCTGCGCGCTCGCTCGCTCACTGAGGCCGCCCGGGCAAAGCCCGGGCGTCGGGCGACCTTTGGTCGCCC
GGCCTCAGTGAGCGAGCGAGCGCGCAGAGAGGGAGTGGCCAACTCCATCACTAGGGGTTCCTTGTAGTTA
ATGATTAACCCGCCATGCTACTTATCTACGTAGCCATGCTCTAGGTCGTTACATAACTTACGGTAAATGGC
CCGCCTGGCTGACCGCCCAACGACCCCCGCCCATTGACGTCAATAATGACGTATGTTCCCATAGTAACGCC
AATAGGGACTTTCCATTGACGTCAATGGGTGGAGTATTTACGGTAAACTGCCCACTTGGCAGTACATCAAG
TGTATCATATGCCAAGTACGCCCCCTATTGACGTCAATGACGGTAAATGGCCCGCCTGGCATTATGCCCAG
TACATGACCTTATGGGACTTTCCTACTTGGCAGTACATCTACGTATTAGTCATCGCTATTACCATGGTGATG
CGGTTTTGGCAGTACATCAATGGGCGTGGATAGCGGTTTGACTCACGGGGATTTCCAAGTCTCCACCCCAT
TGACGTCAATGGGAGTTTGTTTTGGCACCAAAATCAACGGGACTTTCCAAAATGTCGTAACAACTCCGCCC
CATTGACGCAAATGGGCGGTAGGCGTGTACGGTGGGAGGTCTATATAAGCAGAGCTCGTTTAGTGAACCG
TCAGATCGCCTGGAGACGCCATCCACGCTGTTTTGACCTCCATAGAAGACACCGGGACCGATCCAGCCTCC
GGACTCTAGAGGATCCGGTACTCGAGGAACTGAAAAACCAGAAAGTTAACTGGTAAGTTTAGTCTTTTTGT
CTTTTATTTCAGGTCCCGGATCCGGTGGTGGTGCAAATCAAAGAACTGCTCCTCAGTGGATGTTGCCTTTAC
TTCTAGGCCTGTACGGAAGTGTTACTTCTGCTCTAAAAGCTGCGGAATTGTACCCGCGGCCGCGAATTCAG
AGCCGCCACCATGAAGCACCTGTGGTTCTTCCTGCTGCTGGTGGCAGCACCAAGATGGGTGCTGAGCGAG
GTGCAGCTGGTGGAGTCTGGAGGAGGACTGGTGAAGCCAGGAGGAAGCCTGAGGCTGTCTTGCGCCGCCT
CCGGGTACACCTTCTCCAACTACTGGATCGGATGGGTGAGACAGGCACCAGGCAAGGGACTGGAGTGGAT
CGGCGACATCTACCCCGGAGGCAACTACATCCGCAACAACGAGAAGTTCAAGGACAAGACCACCCTGAGC
GCCGACACCTCTAAGAACACCGCCTACCTGCAGATGAACTCTCTGAAGACCGAGGACACCGCCGTGTACT
ACTGCGGATCCAGCTTCGGCTCCAACTACGTGTTCGCCTGGTTCACCTACTGGGGACAGGGCACCCTGGTG
ACCGTGTCTTCCGCCAGCACCAAGGGACCATCCGTGTTCCCACTGGCATCCTGCAGCAGGTCTACCTCCGA
GAGCACCGCCGCCCTGGGATGCCTGGTGAAGGACTACTTCCCTGAGCCAGTGACCGTGTCTTGGAACTCCG
GGGCCCTGACCTCCGGAGTGCACACCTTCCCAGCCGTGCTGCAGAGCTCTGGACTGTACAGCCTGTCCAGC
GTGGTGACCGTGCCTTCTTCCAGCCTGGGCACCCAGACCTACGTGTGCAACGTGGTGCACGAGCCAAGCAA
CACCAAGGTGGACAAGAGGGTGGAGTTCACCCCACCTTGCCCACCATGCCCAGCACCTGAGGCAGCAGGG
GGACCCTCCGTGTTCCTGTTCCCTCCAAAGCCTAAGGACACCCTGATGATCTCTCGCACCCCAGAGGTGAC
CTGCGTGGTGGTGGACGTGTCCCAGGAGGACCCCGAGGTGCAGTTCAACTGGTACGTGGACGGGGTGGAG
GTGCACAACGCACAGACCAAGCCAAGGGAGCGCCAGTTCAACTCCACCTACAGGGTGGTGAGCGTGCTGA
CCGTGACCCACCAGGACTGGCTGAACGGCAAGGAGTACACCTGCAAGGTGAGCAACAAGGGGCTGCCAG
CCCCCATCGAGAAGACCATCTCTAAGGCCAAGGGACAGCCCAGAGAGCCTCAGGTGTACATCCTGCCCCC
TCCACAGGAGGAGCTGACCAAGAACCAGGTGAGCCTGACCTGCCTGGTGACCGGCTTCTACCCTAGCGAC
ATCGCCGTGGAGTGGGAGTCTAACGGGCAGCCAGAGAACACCTACAAGACCACCCCACCTGTGCTGGACA
GCGACGGGTCTTACCTGCTGTACTCCAAGCTGACCGTGAACAAGAGCAGGTGGCAGCCTGGAAACATCTT
CACCTGCTCCGTGCTGCACGAGGCCCTGCACAGCCACTACACTCAGAAGTCTCTGTCCGTGAGCCCAGGCA
AGAGAAAGAGGAGATCTGGGTCCGGAGCCACCAACTTCAGCCTGCTGAAGCAGGCAGGAGACGTGGAGG
AGAACCCTGGACCAATGAAACACCTGTGGTTCTTCCTGTTACTCGTCGCCGCCCCAAGATGGGTGCTGTCC
GACATCGTGATGACCCAGTCCCCTCTGAGCCTGCCAGTGACCCCAGGAGAGCCTGCCAGCATCTCTTGCAG
GTCTTCCCAGAGACTGCTGAGCTCTTACGGACACACCTACCTGCACTGGTACCTGCAGAAGCCTGGCCAGA
GCCCACAGCTGCTGATCTACGAGGTGTCCAACCGGTTCAGCGGAGTGCCAGACCGCTTCTCCGGAAGCGG
GTCTGGAACCGACTTCACCCTGAAGATCTCCAGGGTGGAGGCAGAGGACGTGGGCGTGTACTACTGCTCC
CAGAGCACCCACGTGCCACTGACCTTCGGCCAGGGGACCAAGGTGGAGATCAAGAGGGCAGTGGCAGCA
CCTTCCGTGTTCATCTTCCCACCCTCCGAGGACCAGGTGAAGTCTGGCACCGTGTCCGTGGTGTGCCTGCTG
AACAACTTCTACCCCAGGGAGGCCTCCGTGAAGTGGAAGGTGGACGGCGTGCTGAAGACCGGGAACTCTC
AGGAGTCCGTGACCGAGCAGGACTCTAAGGACAACACCTACTCCCTGTCCAGCACCCTGACCCTGTCTTCC
ACCGACTACCAGAGCCACAACGTGTACGCATGCGAGGTGACCCACCAGGGACTGAGCTCTCCTGTGACCA
AGTCTTTCAACAGAGGAGAGTGCTGAGCGGCCGCGAATTCACTAGTTCCATAAAGTAGGAAACACTACAT
CCATAAAGTAGGAAACACTACATCCATAAAGTAGGAAACACTACATCCATAAAGTAGGAAACACTACAGC
TAGCCGCATTATTACTCACGGTACGACGCATTATTACTCACGGTACGACGCATTATTACTCACGGTACGAC
GCATTATTACTCACGGTACGACTTAAGACCGGTGGGGATCCAGACATGATAAGATACATTGATGAGTTTGG
ACAAACCACAACTAGAATGCAGTGAAAAAAATGCTTTATTTGTGAAATTTGTGATGCTATTGCTTTATTTG
TAACCATTATAAGCTGCAATAAACAAGTTAACAACAACAATTGCATTCATTTTATGTTTCAGGTTCAGGGG
GAGGTGTGGGAGGTTTTTTCGGATCCTCTAGAGTCGACCAGAGCATGGCTACGTAGATAAGTAGCATGGC
GGGTTAATCATTAACTACAAGGAACCCCTAGTGATGGAGTTGGCCACTCCCTCTCTGCGCGCTCGCTCGCT
CACTGAGGCCGGGCGACCAAAGGTCGCCCGACGCCCGGGCTTTGCCCGGGCGGCCTCAGTGAGCGAGCGA
GCGCGCAGCTG
>macaque IgG4 LS leronlimab heavy chain nucleotide sequence
SEQ ID NO: 17
GAGGTGCAGCTGGTGGAGTCTGGAGGAGGACTGGTGAAGCCAGGAGGAAGCCTGAGGCTGTCTTGCGCCG
CCTCCGGGTACACCTTCTCCAACTACTGGATCGGATGGGTGAGACAGGCACCAGGCAAGGGACTGGAGTG
GATCGGCGACATCTACCCCGGAGGCAACTACATCCGCAACAACGAGAAGTTCAAGGACAAGACCACCCTG
AGCGCCGACACCTCTAAGAACACCGCCTACCTGCAGATGAACTCTCTGAAGACCGAGGACACCGCCGTGT
ACTACTGCGGATCCAGCTTCGGCTCCAACTACGTGTTCGCCTGGTTCACCTACTGGGGACAGGGCACCCTG
GTGACCGTGTCTTCCGCCAGCACCAAGGGACCATCCGTGTTCCCACTGGCATCCTGCAGCAGGTCTACCTC
CGAGAGCACCGCCGCCCTGGGATGCCTGGTGAAGGACTACTTCCCTGAGCCAGTGACCGTGTCTTGGAACT
CCGGGGCCCTGACCTCCGGAGTGCACACCTTCCCAGCCGTGCTGCAGAGCTCTGGACTGTACAGCCTGTCC
AGCGTGGTGACCGTGCCTTCTTCCAGCCTGGGCACCCAGACCTACGTGTGCAACGTGGTGCACGAGCCAAG
CAACACCAAGGTGGACAAGAGGGTGGAGTTCACCCCACCTTGCCCACCATGCCCAGCACCTGAGGCAGCA
GGGGGACCCTCCGTGTTCCTGTTCCCTCCAAAGCCTAAGGACACCCTGATGATCTCTCGCACCCCAGAGGT
GACCTGCGTGGTGGTGGACGTGTCCCAGGAGGACCCCGAGGTGCAGTTCAACTGGTACGTGGACGGGGTG
GAGGTGCACAACGCACAGACCAAGCCAAGGGAGCGCCAGTTCAACTCCACCTACAGGGTGGTGAGCGTGC
TGACCGTGACCCACCAGGACTGGCTGAACGGCAAGGAGTACACCTGCAAGGTGAGCAACAAGGGGCTGCC
AGCCCCCATCGAGAAGACCATCTCTAAGGCCAAGGGACAGCCCAGAGAGCCTCAGGTGTACATCCTGCCC
CCTCCACAGGAGGAGCTGACCAAGAACCAGGTGAGCCTGACCTGCCTGGTGACCGGCTTCTACCCTAGCG
ACATCGCCGTGGAGTGGGAGTCTAACGGGCAGCCAGAGAACACCTACAAGACCACCCCACCTGTGCTGGA
CAGCGACGGGTCTTACCTGCTGTACTCCAAGCTGACCGTGAACAAGAGCAGGTGGCAGCCTGGAAACATC
TTCACCTGCTCCGTGCTGCACGAGGCCCTGCACAGCCACTACACTCAGAAGTCTCTGTCCGTGAGCCCAGG
CAAG
>macaque IgG4 LS leronlimab light chain nucleotide sequence
SEQ ID NO: 18
GACATCGTGATGACCCAGTCCCCTCTGAGCCTGCCAGTGACCCCAGGAGAGCCTGCCAGCATCTCTTGCAG
GTCTTCCCAGAGACTGCTGAGCTCTTACGGACACACCTACCTGCACTGGTACCTGCAGAAGCCTGGCCAGA
GCCCACAGCTGCTGATCTACGAGGTGTCCAACCGGTTCAGCGGAGTGCCAGACCGCTTCTCCGGAAGCGG
GTCTGGAACCGACTTCACCCTGAAGATCTCCAGGGTGGAGGCAGAGGACGTGGGCGTGTACTACTGCTCC
CAGAGCACCCACGTGCCACTGACCTTCGGCCAGGGGACCAAGGTGGAGATCAAGAGGGCAGTGGCAGCA
CCTTCCGTGTTCATCTTCCCACCCTCCGAGGACCAGGTGAAGTCTGGCACCGTGTCCGTGGTGTGCCTGCTG
AACAACTTCTACCCCAGGGAGGCCTCCGTGAAGTGGAAGGTGGACGGCGTGCTGAAGACCGGGAACTCTC
AGGAGTCCGTGACCGAGCAGGACTCTAAGGACAACACCTACTCCCTGTCCAGCACCCTGACCCTGTCTTCC
ACCGACTACCAGAGCCACAACGTGTACGCATGCGAGGTGACCCACCAGGGACTGAGCTCTCCTGTGACCA
AGTCTTTCAACAGAGGAGAGTGC
>macaque IgG4 LS leronlimab heavy chain protein sequence with signal
peptide (in bold)-CDRs underlined
SEQ ID NO: 19
MKHLWFFLLLVAAPRWVLSEVQLVESGGGLVKPGGSLRLSCAASGYTFSNYWIGWVRQAPGKGLEWIGDIY
PGGNYIRNNEKFKDKTTLSADTSKNTAYLQMNSLKTEDTAVYYCGSSFGSNYVFAWFTYWGQGTLVTVSSAS
TKGPSVFPLASCSRSTSESTAALGCLVKDYFPEPVTVSWNSGALTSGVHTFPAVLQSSGLYSLSSVVTVPSSSLG
TQTYVCNVVHEPSNTKVDKRVEFTPPCPPCPAPEAAGGPSVFLFPPKPKDTLMISRTPEVTCVVVDVSQEDPEV
QFNWYVDGVEVHNAQTKPRERQFNSTYRVVSVLTVTHQDWLNGKEYTCKVSNKGLPAPIEKTISKAKGQPRE
PQVYILPPPQEELTKNQVSLTCLVTGFYPSDIAVEWESNGQPENTYKTTPPVLDSDGSYLLYSKLTVNKSRWQP
GNIFTCSVLHEALHSHYTQKSLSVSPGK
>macaque IgG4 LS leronlimab light chain protein sequence with signal
peptide (in bold)-CDRs underlined
SEQ ID NO: 20
MKHLWFFLLLVAAPRWVLSDIVMTQSPLSLPVTPGEPASISCRSSQRLLSSYGHTYLHWYLQKPGQSPQLLIY
EVSNRFSGVPDRFSGSGSGTDFTLKISRVEAEDVGVYYCSQSTHVPLTFGQGTKVEIKRAVAAPSVFIFPPSEDQ
VKSGTVSVVCLLNNFYPREASVKWKVDGVLKTGNSQESVTEQDSKDNTYSLSSTLTLSSTDYQSHNVYACEVT
HQGLSSPVTKSFNRGEC
>macaque IgG4 LS leronlimab heavy chain nucleotide sequence with
signal peptide sequence (bold)
SEQ ID NO: 21
ATGAAGCACCTGTGGTTCTTCCTGCTGCTGGTGGCAGCACCAAGATGGGTGCTGAGCGAGGTGCAGC
TGGTGGAGTCTGGAGGAGGACTGGTGAAGCCAGGAGGAAGCCTGAGGCTGTCTTGCGCCGCCTCCGGGTA
CACCTTCTCCAACTACTGGATCGGATGGGTGAGACAGGCACCAGGCAAGGGACTGGAGTGGATCGGCGAC
ATCTACCCCGGAGGCAACTACATCCGCAACAACGAGAAGTTCAAGGACAAGACCACCCTGAGCGCCGACA
CCTCTAAGAACACCGCCTACCTGCAGATGAACTCTCTGAAGACCGAGGACACCGCCGTGTACTACTGCGG
ATCCAGCTTCGGCTCCAACTACGTGTTCGCCTGGTTCACCTACTGGGGACAGGGCACCCTGGTGACCGTGT
CTTCCGCCAGCACCAAGGGACCATCCGTGTTCCCACTGGCATCCTGCAGCAGGTCTACCTCCGAGAGCACC
GCCGCCCTGGGATGCCTGGTGAAGGACTACTTCCCTGAGCCAGTGACCGTGTCTTGGAACTCCGGGGCCCT
GACCTCCGGAGTGCACACCTTCCCAGCCGTGCTGCAGAGCTCTGGACTGTACAGCCTGTCCAGCGTGGTGA
CCGTGCCTTCTTCCAGCCTGGGCACCCAGACCTACGTGTGCAACGTGGTGCACGAGCCAAGCAACACCAA
GGTGGACAAGAGGGTGGAGTTCACCCCACCTTGCCCACCATGCCCAGCACCTGAGGCAGCAGGGGGACCC
TCCGTGTTCCTGTTCCCTCCAAAGCCTAAGGACACCCTGATGATCTCTCGCACCCCAGAGGTGACCTGCGT
GGTGGTGGACGTGTCCCAGGAGGACCCCGAGGTGCAGTTCAACTGGTACGTGGACGGGGTGGAGGTGCAC
AACGCACAGACCAAGCCAAGGGAGCGCCAGTTCAACTCCACCTACAGGGTGGTGAGCGTGCTGACCGTGA
CCCACCAGGACTGGCTGAACGGCAAGGAGTACACCTGCAAGGTGAGCAACAAGGGGCTGCCAGCCCCCAT
CGAGAAGACCATCTCTAAGGCCAAGGGACAGCCCAGAGAGCCTCAGGTGTACATCCTGCCCCCTCCACAG
GAGGAGCTGACCAAGAACCAGGTGAGCCTGACCTGCCTGGTGACCGGCTTCTACCCTAGCGACATCGCCG
TGGAGTGGGAGTCTAACGGGCAGCCAGAGAACACCTACAAGACCACCCCACCTGTGCTGGACAGCGACGG
GTCTTACCTGCTGTACTCCAAGCTGACCGTGAACAAGAGCAGGTGGCAGCCTGGAAACATCTTCACCTGCT
CCGTGCTGCACGAGGCCCTGCACAGCCACTACACTCAGAAGTCTCTGTCCGTGAGCCCAGGCAAG
>macaque IgG4 LS leronlimab light chain nucleotide sequence with
signal peptide sequence (bold)
SEQ ID NO: 22
ATGAAACACCTGTGGTTCTTCCTGTTACTCGTCGCCGCCCCAAGATGGGTGCTGTCCGACATCGTGAT
GACCCAGTCCCCTCTGAGCCTGCCAGTGACCCCAGGAGAGCCTGCCAGCATCTCTTGCAGGTCTTCCCAGA
GACTGCTGAGCTCTTACGGACACACCTACCTGCACTGGTACCTGCAGAAGCCTGGCCAGAGCCCACAGCTG
CTGATCTACGAGGTGTCCAACCGGTTCAGCGGAGTGCCAGACCGCTTCTCCGGAAGCGGGTCTGGAACCG
ACTTCACCCTGAAGATCTCCAGGGTGGAGGCAGAGGACGTGGGCGTGTACTACTGCTCCCAGAGCACCCA
CGTGCCACTGACCTTCGGCCAGGGGACCAAGGTGGAGATCAAGAGGGCAGTGGCAGCACCTTCCGTGTTC
ATCTTCCCACCCTCCGAGGACCAGGTGAAGTCTGGCACCGTGTCCGTGGTGTGCCTGCTGAACAACTTCTA
CCCCAGGGAGGCCTCCGTGAAGTGGAAGGTGGACGGCGTGCTGAAGACCGGGAACTCTCAGGAGTCCGTG
ACCGAGCAGGACTCTAAGGACAACACCTACTCCCTGTCCAGCACCCTGACCCTGTCTTCCACCGACTACCA
GAGCCACAACGTGTACGCATGCGAGGTGACCCACCAGGGACTGAGCTCTCCTGTGACCAAGTCTTTCAAC
AGAGGAGAGTGC

1. A recombinant adeno-associated virus (rAAV) vector comprising a heterologous nucleic acid encoding a CCR5 antibody comprising the following human IgG Fc amino acid substitutions:
(i) M428L and N434S;
(ii) L234A and L235A; and
(iii) S131C
wherein the antibody comprises:
(a) a heavy chain variable region (VH) comprising a heavy chain complementary determining region 1 (HCDR1) of SEQ ID NO:11, a heavy chain complementary determining region 2 (HCDR2) of SEQ ID NO:12, and a heavy chain complementary determining region 3 (HCDR3) of SEQ ID NO:13; and
(b) a light chain variable region (VL) comprising a light chain complementary determining region 1 (LCDR1) of SEQ ID NO:9, a light chain complementary determining region 2 (LCDR2) of SEQ ID NO: 10, and a light chain complementary determining region 3 (LCDR3) of SEQ ID NO:11.

2. The vector of claim 1, wherein the human IgG Fc is a human IgG1 or IgG4.

3. The vector of claim 1, wherein the vector is a rAAV having a capsid selected from AAV1, AAV2, AAV3, AAV4, AAV5, AAV6, AAV7, AAV8, AAV9, AAV10, AAV11, or AAVrh10 and variants thereof.

4. A method of treating a HIV infection in a subject comprising administering to the subject an effective amount of a recombinant adeno-associated virus (rAAV) vector comprising a heterologous nucleic acid encoding a CCR5 antibody comprising the following human IgG Fc amino acid substitutions:
(i) M428L and N434S;
(ii) L234A and L235A; and
(iii) S131C
wherein the antibody comprises:
(a) a heavy chain variable region (VH) comprising a heavy chain complementary determining region 1 (HCDR1) of SEQ ID NO:11, a heavy chain complementary determining region 2 (HCDR2) of SEQ ID NO:12, and a heavy chain complementary determining region 3 (HCDR3) of SEQ ID NO:13; and
(b) a light chain variable region (VL) comprising a light chain complementary determining region 1 (LCDR1) of SEQ ID NO:9, a light chain complementary determining region 2 (LCDR2) of SEQ ID NO: 10, and a light chain complementary determining region 3 (LCDR3) of SEQ ID NO:11.

5. The method of claim 4, wherein the vector is administered at a dose of at least 1×10¹² genomes/kg.

6. The method of claim 4, wherein the vector is administered at a dose of at least 2×10¹² genomes/kg.

7. The method of claim 4, wherein the percentage of CCR5 receptors occupied by the CCR5 antibody on CCR5+ CD4+ T cells is increased.

8. The method of claim 4, wherein the period of time in which CCR5 receptors are occupied by the CCR5 antibody on CCR5+ CD4+ T cells is extended.

9. The method of claim 1, wherein the period of time in which CCR5 receptors are fully occupied by the CCR5 antibody on CCR5+ CD4+ T cells in the blood is at least 13 weeks.

10. The method of claim 1, wherein the period of time in which CCR5 receptors are fully occupied by the CCR5 antibody on CCR5+ CD4+ T cells in the blood is at least 38 weeks.

11. The method of claim 1, wherein the period of time in which CCR5 receptors are fully occupied by the CCR5 antibody on CCR5+ CD4+ T cells in the mesenteric lymph nodes is at least 13 weeks.

12. The method of claim 1, wherein the period of time in which CCR5 receptors are fully occupied by the CCR5 antibody on CCR5+ CD4+ T cells in the mesenteric lymph nodes is at least 38 weeks.

13. The method of claim 1, wherein the period of time in which CCR5 receptors are fully occupied by the CCR5 antibody on CCR5+ CD4+ T cells in the spleen is at least 13 weeks.

14. The method of claim 1, wherein the period of time in which CCR5 receptors are fully occupied by the CCR5 antibody on CCR5+ CD4+ T cells in the spleen is at least 38 weeks.

15. The method of claim 4, wherein the period of time in which CCR5 receptors are fully occupied by the CCR5 antibody on CCR5+ CD4+ T cells in the blood is at least 13 weeks.

16. The method of claim 4, wherein the period of time in which CCR5 receptors are fully occupied by the CCR5 antibody on CCR5+ CD4+ T cells in the blood is at least 38 weeks.

17. The method of claim 4, wherein the period of time in which CCR5 receptors are fully occupied by the CCR5 antibody on CCR5+ CD4+ T cells in the mesenteric lymph nodes is at least 13 weeks.

18. The method of claim 4, wherein the period of time in which CCR5 receptors are fully occupied by the CCR5 antibody on CCR5+ CD4+ T cells in the mesenteric lymph nodes is at least 38 weeks.

19. The method of claim 4, wherein the period of time in which CCR5 receptors are fully occupied by the CCR5 antibody on CCR5+ CD4+ T cells in the spleen is at least 13 weeks.

20. The method of claim 4, wherein the period of time in which CCR5 receptors are fully occupied by the CCR5 antibody on CCR5+ CD4+ T cells in the spleen is at least 38 weeks.


SEQUENCE LISTING (22 sequences)

SEQ ID NO: 1 - protein, 131 aa, synthetic construct
MKLPVRLLVLMFWIPASSSDIVMTQSPLSLPVTPGEPASISCRSSQRLLSSYGHTYLHWYLQKPGQSPQLLIYEVSNRFSGVPDRFSGSGSGTDFTLKISRVEAEDVGVYYCSQSTHVPLTFGQGTKVEIK

SEQ ID NO: 2 - DNA, 429 bp, synthetic construct
  source 1..429: mol_type = other DNA
tctagaccaccatgaagttgcctgttaggctgttggtgctgatgttctggattcctgcttccagcagtgatattgtgatgacccaatctccactctccctgcctgtcactcctggagagccagcctccatctcttgcagatctagtcagcgccttctgagcagttatggacatacctatttacattggtacctacagaagccaggccagtctccacagctcctgatctacgaagtttccaaccgattttctggggtcccagacaggttcagtggcagtgggtcagggacagatttcacacttaagatcagtagagtggaggctgaggatgtgggagtttattactgctctcaaagtacacatgttcctctcacgttcggacaggggaccaaggtggaaataaaacgtaagtagtcttctcaactctaga

SEQ ID NO: 3 - protein, 141 aa, synthetic construct
MEWSGVFIFLLSVTAGVHSEVQLVESGGGLVKPGGSLRLSCAASGYTFSNYWIGWVRQAPGKGLEWIGDIYPGGNYIRNNEKFKDKTTLSADTSKNTAYLQMNSLKTEDTAVYYCGSSFGSNYVFAWFTYWGQGTLVTVSS

SEQ ID NO: 4 - DNA, 457 bp, synthetic construct
  source 1..457: mol_type = other DNA
acgcgtccaccatggaatggagcggagtctttatctttctcctgtcagtaactgcaggtgtccactccgaggtgcagctggtggagtctggtggaggcttggtaaagcctggaggttcccttagactctcctgtgcagcctctggttacactttcagtaactattggatcggatgggtccgccaggctccaggcaaagggctggagtggattggcgatatctaccctggagggaactacatcaggaacaatgagaagttcaaggacaagaccaccctgtcagcagatacttccaagaacacagcctatctgcaaatgaacagcctgaaaaccgaggacacagccgtgtattactgtggaagcagcttcggtagtaactacgtgttcgcctggtttacttactggggccaagggactctggtcacagtctcctcaggtgagtccttaaaacctctaga

SEQ ID NO: 5 - protein, 141 aa, synthetic construct
MEWSGVFIFLLSVTAGVHSQVQLVQSGPDVKKPGTSMKMSCKTSGYTFSNYWIGWVRQAPGQGLEWIGDIYPGGNYIRNNEKFKDKTTLTADTSTSTAYMQLGSLRSEDTAVYYCGSSFGSNYVFAWFTYWGQGTLVTVSS

SEQ ID NO: 6 - DNA, 457 bp, synthetic construct
  source 1..457: mol_type = other DNA
tctagaccaccatggaatggagcggggtctttatctttctcctgtcagtaactgcaggtgtccactcccaggtccaactggtgcagtctggacctgatgtgaaaaagcctgggacttcaatgaagatgtcctgcaagacgtctggatacaccttcagtaactattggatcggatgggttaggcaggcgcctggacaaggccttgagtggattggagatatttaccctggagggaactatatcaggaacaatgagaagttcaaggacaagaccacactgacggcagacacatcgaccagcacggcctacatgcaacttggcagcctgagatctgaagacactgccgtctattactgtggaagcagcttcggtagtaactacgtgttcgcctggtttacttactggggccaagggactctggtcacagtctcctcaggtgagtccttaaaacctctaga

SEQ ID NO: 7 - protein, 447 aa, synthetic construct
EVQLVESGGGLVKPGGSLRLSCAASGYTFSNYWIGWVRQAPGKGLEWIGDIYPGGNYIRNNEKFKDKTTLSADTSKNTAYLQMNSLKTEDTAVYYCGSSFGSNYVFAWFTYWGQGTLVTVSSASTKGPSVFPLASCSRSTSESTAALGCLVKDYFPEPVTVSWNSGALTSGVHTFPAVLQSSGLYSLSSVVTVPSSSLGTQTYVCNVVHEPSNTKVDKRVEFTPPCPPCPAPEAAGGPSVFLFPPKPKDTLMISRTPEVTCVVVDVSQEDPEVQFNWYVDGVEVHNAQTKPRERQFNSTYRVVSVLTVTHQDWLNGKEYTCKVSNKGLPAPIEKTISKAKGQPREPQVYILPPPQEELTKNQVSLTCLVTGFYPSDIAVEWESNGQPENTYKTTPPVLDSDGSYLLYSKLTVNKSRWQPGNIFTCSVLHEALHSHYTQKSLSVSPGK

SEQ ID NO: 8 - protein, 219 aa, synthetic construct
DIVMTQSPLSLPVTPGEPASISCRSSQRLLSSYGHTYLHWYLQKPGQSPQLLIYEVSNRFSGVPDRFSGSGSGTDFTLKISRVEAEDVGVYYCSQSTHVPLTFGQGTKVEIKRAVAAPSVFIFPPSEDQVKSGTVSVVCLLNNFYPREASVKWKVDGVLKTGNSQESVTEQDSKDNTYSLSSTLTLSSTDYQSHNVYACEVTHQGLSSPVTKSFNRGEC

SEQ ID NO: 9 - protein, 16 aa, synthetic construct
RSSQRLLSSYGHTYLH

SEQ ID NO: 10 - protein, 7 aa, synthetic construct
EVSNRFS

SEQ ID NO: 11 - protein, 9 aa, synthetic construct
SQSTHVPLT

SEQ ID NO: 12 - protein, 5 aa, synthetic construct
NYWIG

SEQ ID NO: 13 - protein, 17 aa, synthetic construct
DIYPGGNYIRNNEKFKD

SEQ ID NO: 14 - protein, 13 aa, synthetic construct
SFGSNYVFAWFTY

SEQ ID NO: 15 - protein, 352 aa, Homo sapiens [sequence omitted]

SEQ ID NO: 16 - DNA, 3820 bp, synthetic construct
  source 1..3820: mol_type = other DNA
cagctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatctacgtagccatgctctaggtcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccggactctagaggatccggtactcgaggaactgaaaaaccagaaagttaactggtaagtttagtctttttgtcttttatttcaggtcccggatccggtggtggtgcaaatcaaagaactgctcctcagtggatgttgcctttacttctaggcctgtacggaagtgttacttctgctctaaaagctgcggaattgtacccgcggccgcgaattcagagccgccaccatgaagcacctgtggttcttcctgctgctggtggcagcaccaagatgggtgctgagcgaggtgcagctggtggagtctggaggaggactggtgaagccaggaggaagcctgaggctgtcttgcgccgcctccgggtacaccttctccaactactggatcggatgggtgagacaggcaccaggcaagggactggagtggatcggcgacatctaccccggaggcaactacatccgcaacaacgagaagttcaaggacaagaccaccctgagcgccgacacctctaagaacaccgcctacctgcagatgaactctctgaagaccgaggacaccgccgtgtactactgcggatccagcttcggctccaactacgtgttcgcctggttcacctactggggacagggcaccctggtgaccgtgtcttccgccagcaccaagggaccatccgtgttcccactggcatcctgcagcaggtctacctccgagagcaccgccgccctgggatgcctggtgaaggactacttccctgagccagtgaccgtgtcttggaactccggggccctgacctccggagtgcacaccttcccagccgtgctgcagagctctggactgtacagcctgtccagcgtggtgaccgtgccttcttccagcctgggcacccagacctacgtgtgcaacgtggtgcacgagccaagcaacaccaaggtggacaagagggtggagttcaccccaccttgcccaccatgcccagcacctgaggcagcagggggaccctccgtgttcctgttccctccaaagcctaaggacaccctgatgatctctcgcaccccagaggtgacctgcgtggtggtggacgtgtcccaggaggaccccgaggtgcagttcaactggtacgtggacggggtggaggtgcacaacgcacagaccaagccaagggagcgccagttcaactccacctacagggtggtgagcgtgctgaccgtgacccaccaggactggctgaacggcaaggagtacacctgcaaggtgagcaacaaggggctgccagcccccatcgagaagaccatctctaaggccaagggacagcccagagagcctcaggtgtacatcctgccccctccacaggaggagctgaccaagaaccaggtgagcctgacctgcctggtgaccggcttctaccctagcgacatcgccgtggagtgggagtctaacgggcagccagagaacacctacaagaccaccccacctgtgctggacagcgacgggtcttacctgctgtactccaagctgaccgtgaacaagagcaggtggcagcctggaaacatcttcacctgctccgtgctgcacgaggccctgcacagccactacactcagaagtctctgtccgtgagcccaggcaagagaaagaggagatctgggtccggagccaccaacttcagcctgctgaagcaggcaggagacgtggaggagaaccctggaccaatgaaacacctgtggttcttcctgttactcgtcgccgccccaagatgggtgctgtccgacatcgtgatgacccagtcccctctgagcctgccagtgaccccaggagagcctgccagcatctcttgcaggtcttcccagagactgctgagctcttacggacacacctacctgcactggtacctgcagaagcctggccagagcccacagctgctgatctacgaggtgtccaaccggttcagcggagtgccagaccgcttctccggaagcgggtctggaaccgacttcaccctgaagatctccagggtggaggcagaggacgtgggcgtgtactactgctcccagagcacccacgtgccactgaccttcggccaggggaccaaggtggagatcaagagggcagtggcagcaccttccgtgttcatcttcccaccctccgaggaccaggtgaagtctggcaccgtgtccgtggtgtgcctgctgaacaacttctaccccagggaggcctccgtgaagtggaaggtggacggcgtgctgaagaccgggaactctcaggagtccgtgaccgagcaggactctaaggacaacacctactccctgtccagcaccctgaccctgtcttccaccgactaccagagccacaacgtgtacgcatgcgaggtgacccaccagggactgagctctcctgtgaccaagtctttcaacagaggagagtgctgagcggccgcgaattcactagttccataaagtaggaaacactacatccataaagtaggaaacactacatccataaagtaggaaacactacatccataaagtaggaaacactacagctagccgcattattactcacggtacgacgcattattactcacggtacgacgcattattactcacggtacgacgcattattactcacggtacgacttaagaccggtggggatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttcggatcctctagagtcgaccagagcatggctacgtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagctg

SEQ ID NO: 17 - DNA, 1341 bp, synthetic construct
  source 1..1341: mol_type = other DNA
gaggtgcagctggtggagtctggaggaggactggtgaagccaggaggaagcctgaggctgtcttgcgccgcctccgggtacaccttctccaactactggatcggatgggtgagacaggcaccaggcaagggactggagtggatcggcgacatctaccccggaggcaactacatccgcaacaacgagaagttcaaggacaagaccaccctgagcgccgacacctctaagaacaccgcctacctgcagatgaactctctgaagaccgaggacaccgccgtgtactactgcggatccagcttcggctccaactacgtgttcgcctggttcacctactggggacagggcaccctggtgaccgtgtcttccgccagcaccaagggaccatccgtgttcccactggcatcctgcagcaggtctacctccgagagcaccgccgccctgggatgcctggtgaaggactacttccctgagccagtgaccgtgtcttggaactccggggccctgacctccggagtgcacaccttcccagccgtgctgcagagctctggactgtacagcctgtccagcgtggtgaccgtgccttcttccagcctgggcacccagacctacgtgtgcaacgtggtgcacgagccaagcaacaccaaggtggacaagagggtggagttcaccccaccttgcccaccatgcccagcacctgaggcagcagggggaccctccgtgttcctgttccctccaaagcctaaggacaccctgatgatctctcgcaccccagaggtgacctgcgtggtggtggacgtgtcccaggaggaccccgaggtgcagttcaactggtacgtggacggggtggaggtgcacaacgcacagaccaagccaagggagcgccagttcaactccacctacagggtggtgagcgtgctgaccgtgacccaccaggactggctgaacggcaaggagtacacctgcaaggtgagcaacaaggggctgccagcccccatcgagaagaccatctctaaggccaagggacagcccagagagcctcaggtgtacatcctgccccctccacaggaggagctgaccaagaaccaggtgagcctgacctgcctggtgaccggcttctaccctagcgacatcgccgtggagtgggagtctaacgggcagccagagaacacctacaagaccaccccacctgtgctggacagcgacgggtcttacctgctgtactccaagctgaccgtgaacaagagcaggtggcagcctggaaacatcttcacctgctccgtgctgcacgaggccctgcacagccactacactcagaagtctctgtccgtgagcccaggcaag

SEQ ID NO: 18 - DNA, 657 bp, synthetic construct
  source 1..657: mol_type = other DNA
gacatcgtgatgacccagtcccctctgagcctgccagtgaccccaggagagcctgccagcatctcttgcaggtcttcccagagactgctgagctcttacggacacacctacctgcactggtacctgcagaagcctggccagagcccacagctgctgatctacgaggtgtccaaccggttcagcggagtgccagaccgcttctccggaagcgggtctggaaccgacttcaccctgaagatctccagggtggaggcagaggacgtgggcgtgtactactgctcccagagcacccacgtgccactgaccttcggccaggggaccaaggtggagatcaagagggcagtggcagcaccttccgtgttcatcttcccaccctccgaggaccaggtgaagtctggcaccgtgtccgtggtgtgcctgctgaacaacttctaccccagggaggcctccgtgaagtggaaggtggacggcgtgctgaagaccgggaactctcaggagtccgtgaccgagcaggactctaaggacaacacctactccctgtccagcaccctgaccctgtcttccaccgactaccagagccacaacgtgtacgcatgcgaggtgacccaccagggactgagctctcctgtgaccaagtctttcaacagaggagagtgc

SEQ ID NO: 19 - protein, 466 aa, synthetic construct
MKHLWFFLLLVAAPRWVLSEVQLVESGGGLVKPGGSLRLSCAASGYTFSNYWIGWVRQAPGKGLEWIGDIYPGGNYIRNNEKFKDKTTLSADTSKNTAYLQMNSLKTEDTAVYYCGSSFGSNYVFAWFTYWGQGTLVTVSSASTKGPSVFPLASCSRSTSESTAALGCLVKDYFPEPVTVSWNSGALTSGVHTFPAVLQSSGLYSLSSVVTVPSSSLGTQTYVCNVVHEPSNTKVDKRVEFTPPCPPCPAPEAAGGPSVFLFPPKPKDTLMISRTPEVTCVVVDVSQEDPEVQFNWYVDGVEVHNAQTKPRERQFNSTYRVVSVLTVTHQDWLNGKEYTCKVSNKGLPAPIEKTISKAKGQPREPQVYILPPPQEELTKNQVSLTCLVTGFYPSDIAVEWESNGQPENTYKTTPPVLDSDGSYLLYSKLTVNKSRWQPGNIFTCSVLHEALHSHYTQKSLSVSPGK

SEQ ID NO: 20 - protein, 238 aa, synthetic construct
MKHLWFFLLLVAAPRWVLSDIVMTQSPLSLPVTPGEPASISCRSSQRLLSSYGHTYLHWYLQKPGQSPQLLIYEVSNRFSGVPDRFSGSGSGTDFTLKISRVEAEDVGVYYCSQSTHVPLTFGQGTKVEIKRAVAAPSVFIFPPSEDQVKSGTVSVVCLLNNFYPREASVKWKVDGVLKTGNSQESVTEQDSKDNTYSLSSTLTLSSTDYQSHNVYACEVTHQGLSSPVTKSFNRGEC

SEQ ID NO: 21 - DNA, 1398 bp, synthetic construct
  source 1..1398: mol_type = other DNA
atgaagcacctgtggttcttcctgctgctggtggcagcaccaagatgggtgctgagcgaggtgcagctggtggagtctggaggaggactggtgaagccaggaggaagcctgaggctgtcttgcgccgcctccgggtacaccttctccaactactggatcggatgggtgagacaggcaccaggcaagggactggagtggatcggcgacatctaccccggaggcaactacatccgcaacaacgagaagttcaaggacaagaccaccctgagcgccgacacctctaagaacaccgcctacctgcagatgaactctctgaagaccgaggacaccgccgtgtactactgcggatccagcttcggctccaactacgtgttcgcctggttcacctactggggacagggcaccctggtgaccgtgtcttccgccagcaccaagggaccatccgtgttcccactggcatcctgcagcaggtctacctccgagagcaccgccgccctgggatgcctggtgaaggactacttccctgagccagtgaccgtgtcttggaactccggggccctgacctccggagtgcacaccttcccagccgtgctgcagagctctggactgtacagcctgtccagcgtggtgaccgtgccttcttccagcctgggcacccagacctacgtgtgcaacgtggtgcacgagccaagcaacaccaaggtggacaagagggtggagttcaccccaccttgcccaccatgcccagcacctgaggcagcagggggaccctccgtgttcctgttccctccaaagcctaaggacaccctgatgatctctcgcaccccagaggtgacctgcgtggtggtggacgtgtcccaggaggaccccgaggtgcagttcaactggtacgtggacggggtggaggtgcacaacgcacagaccaagccaagggagcgccagttcaactccacctacagggtggtgagcgtgctgaccgtgacccaccaggactggctgaacggcaaggagtacacctgcaaggtgagcaacaaggggctgccagcccccatcgagaagaccatctctaaggccaagggacagcccagagagcctcaggtgtacatcctgccccctccacaggaggagctgaccaagaaccaggtgagcctgacctgcctggtgaccggcttctaccctagcgacatcgccgtggagtgggagtctaacgggcagccagagaacacctacaagaccaccccacctgtgctggacagcgacgggtcttacctgctgtactccaagctgaccgtgaacaagagcaggtggcagcctggaaacatcttcacctgctccgtgctgcacgaggccctgcacagccactacactcagaagtctctgtccgtgagcccaggcaag

SEQ ID NO: 22 - DNA, 714 bp, synthetic construct
  source 1..714: mol_type = other DNA
atgaaacacctgtggttcttcctgttactcgtcgccgccccaagatgggtgctgtccgacatcgtgatgacccagtcccctctgagcctgccagtgaccccaggagagcctgccagcatctcttgcaggtcttcccagagactgctgagctcttacggacacacctacctgcactggtacctgcagaagcctggccagagcccacagctgctgatctacgaggtgtccaaccggttcagcggagtgccagaccgcttctccggaagcgggtctggaaccgacttcaccctgaagatctccagggtggaggcagaggacgtgggcgtgtactactgctcccagagcacccacgtgccactgaccttcggccaggggaccaaggtggagatcaagagggcagtggcagcaccttccgtgttcatcttcccaccctccgaggaccaggtgaagtctggcaccgtgtccgtggtgtgcctgctgaacaacttctaccccagggaggcctccgtgaagtggaaggtggacggcgtgctgaagaccgggaactctcaggagtccgtgaccgagcaggactctaaggacaacacctactccctgtccagcaccctgaccctgtcttccaccgactaccagagccacaacgtgtacgcatgcgaggtgacccaccagggactgagctctcctgtgaccaagtctttcaacagaggagagtgc